(19) United States
(12) Patent Application Publication
(10) Pub. No.: US 2017/0322222 A1
(43) Pub. Date: Nov. 9, 2017

(54) METHODS FOR DETECTING GESTATIONAL DIABETES MELLITUS

(71) Applicants: Universität Basel, Basel (CH); Kantonsspital Aarau AG, Aarau (CH)

(72) Inventors: Stavros GIAGLIS, Basel (CH); Sinuhe HAHN, Liestal (CH); Olav LAPAIRE, Binningen (CH); Paul HASLER, Aarau (CH)

(21) Appl. No.: 15/534,516

(22) PCT Filed: Dec. 10, 2015
(86) PCT No.: PCT/EP2015/079292

§ 371 Date: Jun. 9, 2017

(30) Foreign Application Priority Data
Dec. 11, 2014 (EP) 14197446.9

(51) Int. Cl. G01N 33/68; C12Q 1/68

(52) U.S. Cl. CPC G01N 33/689; C12Q 1/6883; G01N 2800/50; G01N 2800/368; G01N 2333/471; C12Q 2600/158; C12Q 2600/118; G01N 2800/52; G01N 2800/042

(57) ABSTRACT

The present invention relates to methods for diagnosing gestational diabetes mellitus (GDM) in a pregnant female.

### THE FIELD OF THE INVENTION

The present invention relates to methods for diagnosing gestational diabetes mellitus (GDM) in a pregnant female. The present invention further relates to methods for predicting or monitoring the efficacy of a therapeutic agent for use in treating GDM in a pregnant female, methods for the selection of a therapeutic agent for use in treating GDM in a pregnant female and methods for differentiating gestational diabetes mellitus (GDM) from auto-immune type I diabetes (T1DM) and/or from non-auto-immune type II diabetes (T2DM) in a pregnant female.

### BACKGROUND OF THE INVENTION

Gestational diabetes mellitus (GDM) is defined by the occurrence of glucose intolerance manifesting during pregnancy in women with no history of diabetes [1,2]. Although therapies exist to manage GDM [3-6], these pregnancies are associated with a number of fetal and maternal complications. These include fetal macrosomia, necessitating caesarean delivery, as well as an increased risk for adverse pregnancy outcomes such as preeclampsia or hypertension in pregnancy [1,7,8]. Post-partum issues can include neonatal hypoglycaemia, jaundice, respiratory distress syndrome, polycycythemia or hypocalcemia, while affected mothers are at a significantly higher risk developing of type 2 diabetes [1,7,8]. Although GDM has been a recognized clinical concern for almost 5 decades, there is still currently a lack of consensus regarding the screening procedure that should be employed [2,5].

Procedures for the latter range from fasting glucose, random glucose to the more commonly accepted glucose challenge test. Diagnosis is confirmed by an oral glucose tolerance test (OGTT), which itself remains to be globally standardized [2,5]. A further concern is that current screening or diagnostic tests are carried out in the 2nd trimester, thereby perhaps missing an important opportunity for intervention early in pregnancy. There is also the concern that new screening approaches will lead to an over diagnosis of the condition, leading to possible incorrect treatment of normal healthy pregnant women [2,5]. Not only will this lead to increased health care costs, but it may also induce unnecessary parental anxiety. The severity of GDM can be stratified depending on the OGTT test result, which will determine the therapeutic route chosen. In severe cases this may include treatment with metformin or insulin, while mild cases may be manageable by a regimen of diet and exercise [4-6,8-10].

The underlying aetiology of GDM is not clear but is proposed to involve a contra-insulin effect mediated by placentally produced hormones and cytokines, a condition which may be exacerpated by obesity or high caloric diet or inherent genetic disposition prevalent in certain race groups [1,2].

To date there are no reliable screening or diagnostic tests for GDM, in particular no reliable screening or diagnostic tests which can be employed early in pregnancy. Thus there is a need for providing methods for efficient GDM diagnosing tests which can be employed early in pregnancy, e.g. in the 1^{st} trimester.

### SUMMARY OF THE INVENTION

The present invention relates generally to methods for diagnosing gestational diabetes mellitus (GDM) in a pregnant female. The present invention provides methods, which allow diagnosing, predicting, and/or monitoring of GDM and selecting a therapeutic agent for use in treating GDM. Using the methods described herein the level of biomarkers like cell-free DNA, cell-free nucleosomes, myeloperoxidase and/or alpha-1 antitrypsin in the serum or plasma sample isolated from a pregnant female can be used for treatment decisions early in pregnancy.

In a first aspect, the present invention relates to a method for diagnosing gestational diabetes mellitus (GDM) in a pregnant female, said method comprising detecting NETosis in a serum or plasma sample isolated from said female, wherein the level of NETosis in the serum or plasma sample is indicative whether the female is suffering from or whether the female is at risk for developing GDM.

In a further aspect the present invention relates to a method for diagnosing gestational diabetes mellitus (GDM) in a pregnant female, said method comprising detecting citrullinated histone H3 (citH3) in neutrophils isolated from said female, wherein the level of citrullinated histone H3 in the neutrophils is indicative whether the female is suffering from or whether the female is at risk for developing GDM.

In a further aspect the present invention relates to a method for diagnosing gestational diabetes mellitus (GDM) in a pregnant female, said method comprising detecting at least one biomarker selected from the group consisting of cell-free DNA, cell-free nucleosomes, myeloperoxidase and alpha-1 antitrypsin in a serum or plasma sample isolated from said female, wherein the level of at least one of said biomarker in the serum or plasma sample is indicative whether the female is suffering from or whether the female is at risk for developing GDM.

In a further aspect the present invention relates to a method for predicting or monitoring the efficacy of a therapeutic agent for use in treating GDM in a pregnant female, said method comprising detecting the level of at least one biomarker selected from the group consisting of cell-free DNA, cell-free nucleosomes, myeloperoxidase and alpha-1 antitrypsin in a serum or plasma sample isolated from a female pre and post treatment with said therapeutic agent, wherein a change in the level of at least one of said biomarker indicates efficacy of said agent. In a further aspect the present invention relates to a method for differentiating gestational diabetes mellitus (GDM) from auto-immune type I diabetes (T1DM) and/or from non-auto-immune type II diabetes (T2DM) in a pregnant female, said method comprising detecting at least one biomarker selected from the group consisting of cell-free DNA, cell-free nucleosomes, myeloperoxidase, neutrophil elastase, alpha-1 antitrypsin and DNaseI in a serum or plasma sample isolated from said female, wherein the level of at least one of said biomarker in the serum or plasma sample is indicative whether the female is suffering from or whether the female is at risk for developing GDM and/or whether the female is suffering from auto-immune type I diabetes (T1DM) or from non-auto-immune type II diabetes (T2DM). In a further aspect the present invention relates to a method for diagnosing gestational diabetes mellitus (GDM) in a pregnant female, said method comprising detecting the level of polymorphonuclear neutrophils (PMN) and peripheral blood mononuclear cells (PBMC) in a blood sample isolated from said female, wherein the ratio of PMN to PBMC in the blood sample is indicative whether the female is suffering from or whether the female is at risk for developing GDM.

In a further aspect the present invention relates to a method for predicting the risk for developing a disorder selected from the group consisting of preeclampsia, thrombotic events and cardiovascular damage in a pregnant female said method comprising detecting NETosis in a serum or plasma sample isolated from said female, wherein the level of NETosis in the serum or plasma sample is indicative whether said female is at risk for developing preeclampsia, thrombotic events or cardiovascular damage.

In a further aspect the present invention relates to a method for the selection of a therapeutic agent for use in treating GDM in a pregnant female, comprising detecting NETosis in a serum or plasma sample isolated from said female, wherein the level of NETosis in the serum or plasma sample is indicative whether circulatory polymorphic neutrophils (PMN) are used as target for the therapeutic agent.

The present invention further relates to a composition comprising one or more reagents which binds specifically with and/or detects cell-free DNA, cell-free nucleosomes, myeloperoxidase and/or alpha-1 antitrypsin in a serum or plasma sample isolated from a female for use in diagnosing, predicting, and/or monitoring of GDM and/or for selecting a therapeutic agent for use in treating GDM and a kit comprising this composition.

The present invention further relates to a composition comprising one or more reagents which bind specifically with and/or detects citrullinated histone H3 in neutrophils isolated from a female for use in diagnosing, predicting, and/or monitoring of GDM and/or for selecting a therapeutic agent for use in treating GDM and a kit comprising this composition.

The present invention further relates to the use of the method or the kit as described below in diagnosing, predicting, and/or monitoring of GDM and/or in selecting a therapeutic agent for use in treating GDM in a female and the use of cell-free DNA, cell-free nucleosomes, myeloperoxidase, alpha-1 antitrypsin and/or citrullinated histone H3 or the use of cell-free DNA, cell-free nucleosomes, myeloperoxidase and/or alpha-1 antitrypsin, in diagnosing, predicting, and/or monitoring of GDM and/or in selecting a therapeutic agent for use in treating GDM in a female.

### BRIEF DESCRIPTION OF THE FIGURES

FIG. 1 shows the detection of NETosis undergone by isolated circulatory neutrophils over an in-vitro culture period of 3 hours. Neutrophils were isolated by a standard density centrifugation procedure. NETs were detected by fluorescent immunohistochemistry for neutrophil elastase (NE) in green and DNA by staining with DAPI (4′,6-Diamidin-2-phenylindol) in blue. The upper three panels (left to right) indicate that neutrophils isolated from a normal control pregnancy undergo minimal NETosis over the three-hour culture period, in contrast to neutrophils isolated from a case with gestational diabetes mellitus (GDM) (2^{nd} panel, left to right), where NETs are detected at the first time point immediately after isolation. By the three-hour time point large numbers of neutrophils have undergone spontaneous NETosis. The neutrophils isolated from a case with GDM were furthermore exquisitely sensitive to additional stimulation using the phorbol ester PMA (phorbol-12-myristate-13-acetate), bottom panel left to right, where high numbers of NETs are detected at all time points following such treatment.

FIG. 2 indicates the morphometric analysis results of NETs detection by fluorescent immunohistochemistry of isolated neutrophils during a three-hour in-vitro culture. For this analysis samples were obtained from six normal healthy blood donors, as well as from 3 normal pregnancies and a case with GD gestational diabetes mellitus (GDM) during the 2^{nd} (IIT), 3^{rd} (HIT) trimester and post partum (PP—48 hours after delivery). For this analysis, neutrophils were isolated by density gradient centrifugation following venipuncture and cultured in vitro for a period of 3 hours. The presence of NETs were detected by fluorescent immunohistochemistry for the presence of myeloperoxidase (MPO) and DAPI (4′,6-Diamidin-2-phenylindol). These data indicate that the levels of NETs, as enumerated microscopically, were elevated in a case with GDM (lower panel) when compared to matching healthy pregnancies (upper panel) and control non-pregnant individuals for all three phases of pregnancy examined. T1, T2 and T3 refer to the three culture timepoints over the three-hour in-vitro time period, with T1 being representative of the start, T2 being 1 hour after culture initiation and T3 at the end of three-hour culture.

FIG. 3 indicates the analysis of NETosis in samples obtained from six normal healthy blood donors (control), as well as from 3 normal pregnancies (NP) and 3 cases with gestational diabetes mellitus (GDM) during the 2^{nd} (IIT), 3^{rd} (IIIT) trimester and post partum (PP—48 hours after delivery), as determined by the detection of cell-free nucleosomes in culture supernatants. This analysis indicates that neutrophils from GDM cases undergo higher degrees of NETosis than comparable neutrophils from normal healthy pregnancies and healthy non-pregnant controls, when matched for gestational age. In this analysis, neutrophils were isolated by density gradient centrifugation following venipuncture (S-Monovette, Sarstedt) and cultured in vitro for a period of 3 hours. Following each culture time point, NETs were removed by DNaseI treatment, aspiration and the concentration of cell-free nucleosomes was quantified using the Human Cell Death Detection ELISA^{PLUS} Kit® (Roche Diagnostics). NP refers to normal pregnancy, and GDM refers to cases with gestational diabetes mellitus, while control refers to normal non-pregnant individuals. T1, T2 and T3 refer to the three culture timepoints over the three-hour in-vitro time period, with T1 being representative of the start, T2 being 1 hour after culture initiation and T3 at the end of the three-hour culture.

FIG. 4 shows the detection of cell-free DNA in plasma or corresponding serum samples obtained from 10 healthy non-pregnant controls (control), 6 healthy normal pregnancies (NP) and 3 cases with gestational diabetes mellitus (GDM), using blood samples obtained in the 2^{nd} (IIT) and 3^{rd} (HIT) trimesters of pregnancy, and post-partum (PP—48 hours after delivery). Plasma samples were prepared rapidly post venipuncture (S-Monovette, Sarstedt) by centrifugation using standard laboratory procedures and stored frozen at −80° C. till use. Serum samples were allowed to clot at room temperature for one hour, following which it was cleared by centrifugation and the supernatant stored frozen at −80° C. till use. The concentration of cell-free DNA in these plasma or serum samples was determined using a commercial fluorimetric assay employing a DNA binding dye (QuantiFluor® dsDNA System, Promega). This analysis indicates that the levels of cell-free DNA are elevated in the plasma of cases with GDM, when compared to matching healthy pregnancies. This elevation is most pronounced in samples obtained post-partum. In serum samples, a similar pattern is observed in that cell-free DNA levels are elevated in GDM cases, however, in this instance the concentrations are almost 2-fold higher. NP refers to normal pregnancy, and GDM refers to cases with gestational diabetes mellitus, while control refers to normal non-pregnant individuals. IIT, IIIT and PP refer to the second trimester, third trimester and post partum samples, respectively.

FIG. 5 shows the concentration of cell-free nucleosomes in plasma or corresponding serum samples obtained from 10 healthy non-pregnant controls (control), 6 healthy normal pregnancies (NP) and 3 cases with gestational diabetes mellitus (GDM), using blood samples obtained in the 2^{nd} (IIT) and 3^{rd} (HIT) trimesters of pregnancy, and post-partum (PP—48 hours after delivery). Plasma samples were prepared rapidly post venipuncture (S-Monovette, Sarstedt) by centrifugation using standard laboratory procedures and stored frozen at −80° C. till use. Serum samples were allowed to clot at room temperature for one hour, following which it was cleared by centrifugation and the supernatant stored frozen at −80° C. till use. The concentration of cell-free nucleosomes in these plasma or serum samples was determined using a commercial immunoassay (Human Cell Death Detection ELISA^{PLUS} Kit®, Roche Diagnostics). This analysis indicates that the levels of cell-free nucleosomes are elevated in the plasma of cases with GDM, when compared to matching healthy pregnancies. This elevation is most pronounced in samples obtained post-partum. In serum samples, a similar pattern is observed in that cell-free nucleosome levels are elevated in GDM cases, however, in this instance the concentrations are almost 2 fold higher. NP refers to normal pregnancy, and GDM refers to cases with gestational diabetes mellitus, while control refers to normal non-pregnant individuals. IIT, IIIT and PP refer to the second trimester, third trimester and post partum samples, respectively.

FIG. 6 shows that plasma cell-free nucleosome levels are higher in cases with GDM than in the inflammatory condition, rheumatoid arthritis, a condition known to be associated with elevated NETosis. Blood samples were obtained from normal healthy blood donors, normal healthy pregnant women, cases with GDM and those with rheumatoid arthritis. Plasma samples were prepared rapidly post venipuncture (S-Monovette, Sarstedt) by centrifugation using standard laboratory procedures and stored frozen at −80° C. till use. The concentration of cell-free nucleosomes in these plasma samples was determined using a commercial immunoassay (Cell Death ELISA Plus immunoassay (Human Cell Death Detection ELISA^{PLUS} Kit®, Roche Diagnostics). This analysis indicates that the levels of cell-free nucleosomes are elevated in the plasma of cases with GDM, when compared to matching healthy pregnancies, or an inflammatory condition, rheumatoid arthritis. Healthy controls are indicated by Control, healthy pregnancies by Pregnancy, cased with gestational diabetes mellitus by GDM and cases with rheumatoid arthritis by RA.

FIG. 7 shows the concentrations of myeloperoxidase in serum samples obtained from 10 normal healthy blood donors, 6 healthy pregnancies and 3 cases with gestational diabetes mellitus (GDM), using samples obtained in the 2^{nd} (IIT) and 3^{rd} (HIT) trimester, as well as post partum (PP—48 hours after gestation). The concentration of myeloperoxidase was determined using a commercial immunoassay (MPO Human ELISA kit, Hycult Biotech). This analysis indicates that myeloperoxidase levels were greater in GDM samples than from matching normal pregnant women. 2^{nd} trimester is referred to as IIT, the 3^{rd} trimester as IIIT, and 48 hours post partum as PP. Normal pregnancy is referred to as NP, and cases with gestational diabetes mellitus by GDM.

FIG. 8 shows the numbers of peripheral polymorphonuclear neutrophils, peripheral blood mononuclear cells and the ratio of these, in blood samples obtained from 10 normal healthy blood donors, from 6 normal healthy pregnancies in the 2^{nd} (IIT) and 3^{rd} (IIIT) trimester, as well as post partum (PP—48 hours after delivery) and from 3 cases affected by gestational diabetes mellitus (GDM) in the 2^{nd} and 3^{rd} trimester. Venous blood samples were examined rapidly post venepuncture by commercial apparatus (S-Monovette, Sarstedt) to determine the differential count of peripheral polymorphonuclear neutrophils and peripheral blood mononuclear cells. This analysis indicated that the levels of neutrophils was elevated in the circulation of cases with GDM when compared to matching healthy pregnancies, for all three stages examined. These data also indicate that the ratio of neutrophils to peripheral blood mononuclear cells is increased in 2^{nd} and 3^{rd} trimester GDM samples when compared to control pregnancies, but that this returns to normal in post-partum samples. 2^{nd} trimester is referred to as IIT, the 3^{rd} trimester as HIT, and post partum as PP. Normal pregnancy is referred to as NP, and cases with gestational diabetes mellitus by GDM. Polymorphonuclear neutrophils are indicated as PMN, and peripheral blood mononuclear cells as PBMC.

FIG. 9 shows that serum levels of alpha-1 antitrypsin (a1AT) are reduced in the serum of 3 cases with gestational diabetes mellitus (GDM), when compared to 10 normal healthy non-pregnant blood donors, 6 healthy matching pregnancies, as well as 10 cases with type 1 diabetes mellitus (T1DM) and 10 cases with type 2 diabetes mellitus (T2DM). Samples from 6 healthy pregnancies and 3 cases with GDM were obtained in the 2^{nd} (IIT) and 3^{rd} (IIIT) trimester, as well as post partum (PP—48 hours after gestation. Serum samples were allowed to clot at room temperature for one hour, following which it was cleared by centrifugation and the supernatant stored frozen at −80° C. till use. The concentration of alpha-1 antitrypsin (a1AT) in these serum samples was determined using a commercial immunoassay (Human alpha-1 Antitrypsin ELISA kit, Assaypro LLC). These data indicate that serum alpha-1 antitrypsin (a1AT) levels are lower than in comparable pregnancies, those with preeclampsia or non-pregnant individuals with either type 1 diabetes mellitus (T1DM) or type 2 diabetes mellitus (T2DM). Second trimester is referred to as IIT, the 3^{rd} trimester as IIIT, and 48 hours post partum as PP. Cases with gestational diabetes mellitus is referred to by GDM, type 1 diabetes mellitus is referred to by T1DM and type 2 diabetes mellitus by T2DM.

FIG. 10 shows reduced plasma levels of alpha-1 antitrypsin (a1AT) in 3 cases with gestational diabetes mellitus (GDM), when compared to 10 normal healthy non-pregnant blood donors and 6 healthy matching pregnancies. Plasma samples were prepared rapidly post venipuncture (S-Monovette, Sarstedt) by centrifugation using standard laboratory procedures and stored frozen at −80° C. till use. The concentration of alpha-1 antitrypsin (a1AT) in these plasma samples was determined using a commercial immunoassay (Human alpha-1 Antitrypsin ELISA kit, Assaypro LLC). This analysis indicates that the levels of alpha-1 antitrypsin (a1AT) are elevated in the plasma of cases with GDM, when compared to matching healthy pregnancies, for all three phases of pregnancy examined. Second trimester is referred to as IIT, the 3^{rd} trimester as IIIT, and post partum as PP. Normal pregnancy is referred to as NP, and cases with gestational diabetes mellitus by GDM. Additionally in healthy pregnancies, 1^{st} trimester samples were examined, indicated by IT.

FIG. 11 shows levels of DNAse in 10 non-pregnant healthy blood donors, 6 healthy pregnancies, 3 cases with gestational diabetes mellitus (GDM), 10 cases with type 1 diabetes mellitus (T1DM) and 10 cases with type 2 diabetes mellitus (T2DM). In this examination serum samples were obtained from normal healthy blood donors, healthy pregnancies (NP) in the 1^{st} (IT), 2^{nd} (IIT) and 3^{rd} (IIIT) trimester, as well with GDM in the 2^{nd} and 3^{rd} trimester. In all instances a further sample was collected 48 hours post partum (PP). In addition serum was obtained from cases with diagnosed type 1 diabetes mellitus (T1DM) or type 2 diabetes mellitus (T2DM). Serum DNAse levels were determined by a commercial assay (DNase Activity ELISA kit, Orgentec Diagnostica GmbH). This analysis indicates that serum DNAse levels are reduced in cases with type 1 diabetes mellitus (T1DM). In normal pregnancies, as well as in those with GDM, there is a gradual decrease with increase in gestation. In normal pregnancies this decrease is reversed in post aprtum samples, a feature not evident in similar samples from cases with GDM. Normal pregnancy is referred to as NP, and cases with gestational diabetes mellitus by GDM. First trimester is indicated by IT, 2^{nd} trimester by IIT, the 3^{rd} trimester by IIIT, and post partum by PP.

FIG. 12 shows levels of cell-free nucleosomes, serum myeloperoxidase and serum neutrophil elastase in 10 non-pregnant healthy blood donors, 6 healthy pregnancies during the 3^{rd} trimester and post-partum, 3 cases with gestational diabetes mellitus (GDM) during the 3^{rd} trimester and post-partum, 10 cases with type 1 diabetes mellitus (T1DM) and 10 cases with type 2 diabetes mellitus (T2DM). In this examination serum samples were obtained from normal healthy blood donors (control), healthy pregnancies (NP) in the 3^{rd} trimester and 48 hours after delivery (NP PP), as well cases with gestational diabetes mellitus (GDM) in the 3^{rd} trimester and 48 hours after delivery (GDM PP). In addition serum was obtained from cases with diagnosed type 1 diabetes mellitus (T1DM) or type 2 diabetes mellitus (T2DM). Serum cell-free nucleosome levels, MPO levels and NE levels were determined by commercial assays (Human Cell Death Detection ELISA^{PLUS} Kit®, Roche Diagnostics, Human MPO ELISA kit, Hycult Biotech and Elastase/a1-PI Complex ELISA Kit, Calbiochem, respectively). This analysis indicates that the levels of cell-free nucleosome, MPO and NE are elevated in cases with gestational diabetes mellitus (GDM), type 1 diabetes mellitus (T1DM) and type 2 diabetes mellitus (T2DM) compared to healthy non-pregnant blood donors (control). In particular, the cell-free nucleosome levels are elevated in the GDM cases compared to normal pregnancies (NP), which is even more pronounced after delivery. MPO and NE levels show the highest levels in the group of T1DM patients. Normal pregnancy is referred to as NP, cases with gestational diabetes mellitus by GDM, type 1 diabetes mellitus as T1DM, type 2 diabetes mellitus as T2DM, and post partum by PP.

FIG. 13 shows the polymorphonuclear neutrophil (PMN) and peripheral blood mononuclear (PBMC) cell counts (upper panel), as well as the PMN/PBMC ratio (lower panel) during the OGTT are depicted. Venous blood samples were examined rapidly post venepuncture by commercial apparatus (S-Monovette, Sarstedt) to determine the differential count of peripheral polymorphonuclear neutrophils and peripheral blood mononuclear cells. This analysis indicated that GDM patients exhibit a PMN count which is the highest compared to matched non-pregnant controls and control pregnant individuals. Moreover, the PBMC count is the lowest, which leads to distinctively higher levels of the PMN/PBMC ratio in GDM patients compared to matched non-pregnant controls and control pregnant individuals. P refers to normal pregnancy, non-pregnant individuals as non-P and GDM refers to cases with gestational diabetes mellitus, while control refers to normal non-pregnant individuals.

FIG. 14 shows the percentages of polymorphonuclear neutrophil (PMN) and peripheral blood mononuclear (PBMC) cellular populations (upper panel), as well as the PMN/PBMC ratio (lower panel) during the oral glucose tolerance test (OGTT). Again, GDM patients exhibit the highest PMN tendencies compared to the matched non-pregnant controls and control pregnant individuals. Moreover, the percentage of PBMC is the lowest in GDM patients, which again leads to distinctively higher levels of the PMN/PBMC ratio in GDM compared to matched non-pregnant controls and control pregnant individuals. P refers to normal pregnancy, non-pregnant individuals as non-P and GDM refers to cases with gestational diabetes mellitus, while control refers to normal non-pregnant individuals.

FIG. 15 shows the platelet (PLT) differential counts (upper panel), as well as the tendencies concerning morphological characteristics of the platelets (lower panel), such as platelet distribution width (PDW, left) and mean platelet volume (MPV, right) in GDM patients, matched non-pregnant controls and control pregnant individuals during the oral glucose tolerance test (OGTT). In all case values were the lowest in GDM cases compared to matched non-pregnant controls and control pregnant individuals, which indicates possible alterations in the activity of the platelets. P refers to normal pregnancy, non-pregnant individuals as non-P and GDM refers to cases with gestational diabetes mellitus, while control refers to normal non-pregnant individuals.

FIG. 16 shows the intracellular (left panel) and overall (right panel) reactive oxygen species (ROS) generation in neutrophils from GDM patients compared to matched non-pregnant controls and control pregnant individuals the oral glucose tolerance test (OGTT). ROS generation was assessed by 2′-7′-Dichlorodihydro fluorescein diacetate (DCFH-DA) and Luminol fluorimetry, respectively. In all cases, neutrophils from GDM patients produced particularly higher levels of ROS in compared to matched non-pregnant controls and control pregnant individuals, reflecting a higher activation state. P refers to normal pregnancy, non-pregnant individuals as non-P and GDM refers to cases with gestational diabetes mellitus, while control refers to normal non-pregnant individuals.

In FIG. 17 data concerning the morphometric analysis results of NETs detection by fluorescent immunohistochemistry of isolated neutrophils during a three-hour in-vitro culture the oral glucose tolerance test (OGTT) are shown. For this analysis, neutrophils were isolated by density gradient centrifugation following venipuncture and cultured in vitro for a period of 3 hours. The presence of NETs were detected by fluorescent immunohistochemistry for the presence of myeloperoxidase (MPO) and DAPI (4′,6-Diamidin-2-phenylindol). These data indicate that the levels of NETs, as enumerated microscopically, were elevated in a case with GDM when compared to matching healthy pregnancies and control non-pregnant individuals (left panel). Moreover, pregnant women with GDM showed the highest levels of primed neutrophils, which were detected by immunostaining for citrullinated H3 (citH3) and the delobulated diffused nuclear phenotype. P refers to normal pregnancy, non-pregnant individuals as non-P and GDM refers to cases with gestational diabetes mellitus, while control refers to normal non-pregnant individuals.

FIG. 18 shows the concentration of cell-free nucleosomes in serum or corresponding plasma samples obtained from healthy non-pregnant controls (control), healthy normal pregnancies (P) and cases with gestational diabetes mellitus (GDM) during the oral glucose tolerance test (OGTT). Serum samples were allowed to clot at room temperature for one hour, following which it was additionally cleared by centrifugation and the supernatant stored frozen at −80° C. till use. Plasma samples were prepared rapidly post venipuncture (S-Monovette, Sarstedt) by centrifugation using standard laboratory procedures and stored frozen at −80° C. till use. The concentration of cell-free nucleosomes in these plasma or serum samples was determined using a commercial immunoassay (Human Cell Death Detection ELISAPLUS Kit®, Roche Diagnostics). This analysis indicates that the levels of cell-free nucleosomes are elevated almost 3 fold in the serum of cases with GDM, when compared to matching healthy pregnancies. In serum samples, a similar pattern is observed in that cell-free nucleosome levels are elevated in GDM cases, however, in this instance the concentrations are almost 2 fold higher before the administration of the glucose syrup for the OGTT. P refers to normal pregnancy, non-pregnant individuals as non-P and GDM refers to cases with gestational diabetes mellitus, while control refers to normal non-pregnant individuals.

FIG. 19 shows the concentrations of myeloperoxidase (MPO) protein (top left panel) and mRNA (top right panel) expression in neutrophil lysates obtained normal healthy blood donors, healthy pregnancies and cases with gestational diabetes mellitus (GDM), using samples obtained in the 2nd trimester during the oral glucose tolerance test (OGTT). Moreover, neutrophil elastase (NE) mRNA expression was also determined (lower panel) during the OGTT. The expression of myeloperoxidase protein was assessed using Western blot analysis, while the mRNA expression of MPO and NE by Taqman real-time PCR assay. This analysis indicates that myeloperoxidase levels were greater in GDM samples than in matching normal pregnant women. Normal pregnancy is referred to as P, non-pregnant individuals as non-P and cases with gestational diabetes mellitus by GDM, while control refers to normal non-pregnant individuals.

FIG. 20 shows the concentrations of peptidyl arginine deiminase 4 (PADI4) protein (top left panel) and mRNA (top right panel) expression in neutrophil lysates obtained normal healthy blood donors, healthy pregnancies and cases with gestational diabetes mellitus (GDM), using samples obtained in the 2nd trimester during the oral glucose tolerance test (OGTT). Moreover, citrullinated histone 3 (citH3) protein levels were also determined (lower panel) during the OGTT. The expression of PADI4 and citH3 protein was assessed using Western blot analysis, while the mRNA expression of PADI4 by Taqman real-time PCR assay. This analysis indicates that PADI4 protein levels were similar between GDM samples and in matching normal pregnancy samples, despite the fact that PADI4 mRNA expression was higher in GDM patients. Nevertheless, GDM patients showed increased levels of citH3 expression compared to the controls, which is indicative of the increased neutrophil priming towards NET formation. Normal pregnancy is referred to as P, non-pregnant individuals as non-P and cases with gestational diabetes mellitus by GDM, while control refers to normal non-pregnant individuals.

FIG. 21 shows that protein expression of alpha-1 antitrypsin (a1AT) is reduced in neutrophil lysates from cases with gestational diabetes mellitus (GDM), when compared to normal healthy non-pregnant blood donors and healthy matching pregnancies (left panel). Samples from the healthy pregnancies and GDM cases were obtained in the 2nd trimester.

Interestingly, the mRNA expression levels of a1AT are particularly higher in neutrophils from patients with GDM compared to the controls, reflecting an activated cellular state. The protein expression levels of alpha-1 antitrypsin (a1AT) in these samples was determined using a commercial immunoassay (Human alpha-1 Antitrypsin ELISA kit, Assaypro LLC), while the mRNA expression of a1AT by Taqman real-time PCR assay. Normal pregnancy is referred to as P, non-pregnant individuals as non-P and cases with gestational diabetes mellitus by GDM, while control refers to normal non-pregnant individuals.

In FIG. 22 expression levels of IL6, IL8 and TNFa mRNA are shown. mRNA expression of all cytokines were assessed by Taqman real-time PCR assays. This analysis indicates that the expression levels of proinflammatory cytokines are elevated in neutrophils from cases with GDM, when compared to matching healthy pregnancies and non-pregnant healthy donors. Normal pregnancy is referred to as P, non-pregnant individuals as non-P and cases with gestational diabetes mellitus by GDM, while control refers to normal non-pregnant individuals.

### DETAILED DESCRIPTION OF THE INVENTION

The present invention provides methods, which allow diagnosing, predicting, and/or monitoring of GDM and selecting a therapeutic agent for use in treating GDM.

In samples obtained from women pregnant in the 2nd trimester it was surprisingly found that excessively high levels of NETosis occurred. An examination of the patient file revealed the occurrence of GDM in this particular case. It was furthermore observed that the NETosis occurred was caused by polymorphonucleur neutrophil granulocytes (PMN) which constitute the largest population (40-75%) of leucocytes in the human peripheral circulation, and form an important part in the 1st line of defence against microbial pathogens, mainly by phagocytosis or the release of cytotoxic granular enzymes. In subsequent studies these findings have been confirmed in GDM cases in different trimesters (2nd and 3rd), and in samples taken immediately post-partum. This indicates that the finding is reproducible and detectable in the latter part of pregnancy. Nevertheless, it may also occur early in gestation (1^{st} trimester).

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

The term “diagnosing” as used herein means assessing, identifying, evaluating or classifying if a pregnant female suffers from GDM or is at risk for developing GDM. The term “diagnosing” also refers to distinguishing between a pregnant female only bearing risk factors of developing GDM, or already suffering from GDM.

The term “detecting” as used herein refers to methods which include detecting the presence or absence of a substance or the presence or absence of a cellular event like NETosis in a sample and/or qualifying the type of said substance or cellular event. Detecting can be accomplished by methods known in the art and those further described herein. Any suitable method can be used to detect one or more of the biomarkers described herein. These methods include, without limitation, fluorescent DNA bindings dyes (such as QuantiFluor® dsDNA System, Promega), or methods which allow enumeration of DNA molecules by PCR based approaches such as real-time quantitative PCR or digital PCR, massive parallel sequencing (also termed next generation sequencing/NGS) or single molecule real time sequencing (also termed SMRT) approaches, methods using antibodies or aptamers such as ELISA assays which detect e.g. histone molecules or the DNA fragments associated therewith thereby permitting detection of complete nucleosomes, or methods measuring enzyme activity.

The term “predicting” as used herein relates to assessing whether a pregnant female is at risk or not at risk to suffer from GDM. Preferably, it shall be assessed whether a female risk is at elevated risk or at reduced risk as compared to the average risk in a population of subjects.

The term “monitoring” as used herein means that the progression of the disease like GDM (i.e. worsening of the disease) or the regression of the disease (i.e. a patient's recovery) can be made by comparing a measured level of the biomarker to a control or to one or more previous measurements, carried out at different points of time, of the level of the biomarker in the same patient. For example, a decreased level of the biomarker, compared to the result from a previous measurement or to a control may be used to indicate the progression of the disease, while an increased level of the biomarker, compared to the result from a previous measurement or to a control is used to indicate the regression of the disease.

The term “at risk for developing GDM”, as used herein, refers to a pregnant female that has one or more of the following features: an advanced maternal age, ethnicity associated with higher levels of GDM (e.g. African American, Hispanic/Latino American, Pima Indians), high BMI (body mass index) before pregnancy, smoking, large change in weight between pregnancies, short interval between pregnancies, previous amcrosomia, or a family history of type 2 diabetes or GDM, has genetic prevalence for GDM or is classified ‘at risk’ due to lifestyle (e.g. age, diet, general health, occupation, geographical location).

The term “suffering from GDM”, as used herein, refers to a female that displays any degree of glucose intolerance during pregnancy, which resolves shortly after delivery. The current diagnosis is usually made by an examination of fasting glucose levels or following a glucose challenge, termed an oral glucose tolerance test (OGTT).

A biomarker as used herein, refers to a biological compound, such as a protein and a fragment thereof, a peptide, a proteoglycan, a glycoprotein, a lipoprotein, a carbohydrate, a lipid, a nucleic acid, an organic or inorganic chemical, a natural polymer, and a small molecule, which is differentially present in a sample from a subject of one phenotypic status (e.g. having a disease) as compared with another phenotypic status (e.g. not having the disease) and which may be isolated from, or measured in the sample from the subject. The term “fragment” as used herein in relation to a biomarker means a portion of a full-length biomarker protein. Preferred biomarkers of the present invention are selected from the group consisting of cell-free DNA, cell-free nucleosomes, myeloperoxidase, neutrophil elastase, alpha-1 antitrypsin and DNaseI, more preferably cell-free DNA, cell-free nucleosomes, myeloperoxidase, myeloperoxidase in combination with neutrophil elastase, and alpha-1 antitrypsin, even more preferably cell-free DNA, cell-free nucleosomes, myeloperoxidase, or alpha-1 antitrypsin. Equally preferred biomarkers of the present invention are selected from the group consisting of cell-free DNA, cell-free nucleosomes, myeloperoxidase, neutrophil elastase, alpha-1 antitrypsin, citrullinated histone H3 (citH3) and DNaseI, more equally preferably cell-free DNA, cell-free nucleosomes, myeloperoxidase, citrullinated histone H3 or alpha-1 antitrypsin. Preferably two biomarkers, more preferably three biomarkers are detected in the methods of the present invention.

The term “blood sample” refers to a blood sample isolated from the body of a female e.g. a pregnant female. The term “serum or plasma sample” refers to a blood sample isolated from the body of a female e.g. a pregnant female further treated to obtain a serum or plasma sample. The serum or plasma sample can be obtained by methods known in the art such as e.g venepuncture or phlebotomy, using standardized laboratory procedures. Preferably serum samples are used in the methods of the present invention. Preferably, if serum samples are used, no clotting accelerators should be used, as these could lead to erroneous results

The term “control sample” “control blood sample” or “control serum or plasma sample” which are used interchangeably herein refers to an internal or an external control sample, preferably to an external control. An internal control sample is used, i.e. the biomarker level(s) is(are) assessed in the test sample as well as in one or more other sample(s) taken from the same subject to determine if there are any changes in the level(s) of said biomarker(s). An external control sample is used i.e. for an external control sample the presence or concentration of a biomarker in a sample derived from the individual is compared to its presence or concentration in an individual known to suffer from, or known to be at risk of, GDM; or, preferably, is compared to its presence or concentration in an individual known to be free of GDM, i.e., a “healthy female”. Usually the sample's biomarker level is directly or indirectly correlated with a diagnosis and the biomarker level is e.g. used to determine whether an individual is at risk for GDM. Depending on the intended diagnostic use an appropriate control sample is chosen and a control or reference value for the biomarker established therein. It will be appreciated by the skilled artisan that such control sample is usually obtained from a reference population that is age-matched and free of confounding diseases. As also clear to the skilled artisan, the absolute biomarker values established in a control sample will be dependent on the assay used. Preferably samples from 100 well-characterized individuals from the appropriate reference population are used to establish a control (reference) value. Also preferred the reference population may be chosen to consist of 20, 30, 50, 200, 500 or 1000 individuals. Healthy individuals such as healthy females represent a preferred reference population for establishing a control value, i.e. for establishing an external control value. Equally a pregnant female more preferably a healthy pregnant female may serve as external control.

The term “level” as used herein refers to an amount (e.g. relative amount or concentration) of biomarker that is detectable or measurable in a test sample and/or a control sample. The control biomarker level, can for example, be the average or median level in a plurality of normal reference biological samples (e.g. serum or plasma samples from healthy females i.e. from females known not to suffer from and not being at risk for developing GDM). According to the explanation of the terms “lower level” or “higher level”, as used in the context of the present invention, preferably, an increase or decrease of at least 10%, of at least 20%, preferably at least 30%, of the level of the biomarker in the test sample, compared to the level of the biomarker in a control sample from a healthy female is meant.

The term “cut-off level” refers to a tolerance zone for a particular assay, values above or below of which for a subject a sample suggest that it is associated with a particular pathological phenotype. This value is usually determined by the analysis of normal reference samples, and may be specific for a particular laboratory.

The term “NETosis” as used herein refers to the process whereby chromatin and granular components are extruded in the form of neutrophil extracellular traps into the extracellular environment by polymorphonuclear neutrophils

NETosis can be usually detected by biomarkers and/or cellular events such as morphometric analysis of changes undergone by isolated PMN in in vitro culture. Markers for detection include cell-free DNA, cell-free nucleosomes, myeloperoxidase, neutrophil elastase and alpha-1 antitrypsin and further include histones, histones modified by citrullination, in conjunction with neutrophil granular proteins such as myeloperoxidase.

The term “about” as used herein refers to +/−5% of a given measurement.

The methods for diagnosing, predicting, and/or monitoring of GDM in a pregnant female, detecting biomarkers and/or selecting a therapeutic agent for use in treating GDM in a female provided by the present invention are usually in vitro methods. The female is preferably pregnant in the 1^{st}, 2^{nd} or 3^{rd} trimester, more preferably in the 1^{st} or 2^{nd}. The methods provided by the present invention allow for diagnosing, predicting, and/or monitoring of GDM in a pregnant female already at early stage such as in the 1^{st} and/or 2^{nd} trimester of pregnancy. In the context of the present invention the 1^{st} trimester of pregnancy comprises of weeks 0-13 weeks, the 2^{nd} trimester of pregnancy comprises of weeks 14-26, and the 3^{rd} trimester of pregnancy comprises of weeks 27 to 40.

In some embodiments a method for diagnosing gestational diabetes mellitus (GDM) in a pregnant female is provided, said method comprising detecting NETosis in a serum or plasma sample isolated from said female, wherein the level of NETosis in the serum or plasma sample is indicative whether the female is suffering from or whether the female is at risk for developing GDM. NETosis can be detected in a serum or plasma sample e.g. by detecting the level of biomarkers, or by microscopic analysis of changes undergone by isolated PMN in in vitro culture, by examination of PMN cultures for NETs products.

Usually NETosis is detected by detecting the level of at least one biomarker. The at least biomarker is preferably selected from the group consisting of cell-free DNA, cell-free nucleosomes, myeloperoxidase and alpha-1 antitrypsin in the serum or plasma sample isolated from the female.

In some embodiments a method for diagnosing gestational diabetes mellitus (GDM) in a pregnant female is provided, said method comprising detecting at least one biomarker selected from the group consisting of cell-free DNA, cell-free nucleosomes, myeloperoxidase and alpha-1 antitrypsin in a serum or plasma sample isolated from said female, wherein the level of at least one of said biomarker in the serum or plasma sample is indicative whether the female is suffering from or whether the female is at risk for developing GDM.

In some embodiments a method for diagnosing gestational diabetes mellitus (GDM) in a pregnant female is provided, said method comprising detecting at least one biomarker selected from the group consisting of cell-free DNA, cell-free nucleosomes, myeloperoxidase and alpha-1 antitrypsin in combination with neutrophil elastase, preferably myeloperoxidase in combination with neutrophil elastase, in a serum or plasma sample isolated from said female, wherein the level of at least one of said biomarker in the serum or plasma sample is indicative whether the female is suffering from or whether the female is at risk for developing GDM.

In some embodiments the level of the at least one biomarker in the serum or plasma sample isolated from the female to be tested (test sample) is compared to a control serum or plasma sample isolated from a healthy female, wherein a higher level of cell-free DNA, cell-free nucleosomes and/or myeloperoxidase in the serum or plasma sample isolated from said female compared to the control serum or plasma sample is indicative that the female is suffering from or is at risk for developing GDM, and wherein a lower level of alpha-1 antitrypsin in the serum or plasma sample isolated from said female compared to the control serum or plasma sample is indicative that said female is suffering from or is at risk for developing GDM.

In some embodiments the level of citrullinated histone H3 in neutrophils isolated from the female to be tested (test sample) is compared to the level of citrullinated histone H3 in neutrophils of a control sample isolated from a healthy female, wherein a higher level of citrullinated histone H3 in neutrophils isolated from said female compared to the level of citrullinated histone H3 in neutrophils of the control sample is indicative that said female is suffering from or is at risk for developing GDM.

In some embodiments the level of the at least one biomarker in the serum or plasma sample isolated from the female is compared to a cut-off level, wherein if the level of cell-free DNA in serum isolated from the female is higher than the cut-off level, this is indicative that the female is suffering from or is at risk for developing GDM;

wherein if the level of cell-free DNA in plasma isolated from said female is higher than the cut-off level, this is indicative that said female is suffering from or is at risk for developing GDM;
wherein if the level of cell-free nucleosomes in serum isolated from said female is higher than the cut-off level, this is indicative that said female is suffering from or is at risk for developing GDM;
wherein if the level of cell-free nucleosomes in plasma isolated from said female is higher than the cut-off level, this is indicative that said female is suffering from or is at risk for developing GDM;
wherein if the level of myeloperoxidase in serum isolated from said female is higher than the cut-off level, this is indicative that said female is suffering from or is at risk for developing GDM;
wherein if the level of myeloperoxidase in plasma isolated from said female is higher than the cut-off level, this is indicative that said female is suffering from or is at risk for developing GDM;
wherein if the level of alpha-1 antitrypsin in serum isolated from said female is lower than the cut-off level, this is indicative that said female is suffering from or is at risk for developing GDM; and/or
wherein if the level of alpha-1 antitrypsin in plasma isolated from said female is lower than the cut-off level, this is indicative that said female is suffering from or is at risk for developing GDM.

In some embodiments the level of citrullinated histone H3 in neutrophils isolated from the female is compared to a cut-off level, wherein if the level of citrullinated histone H3 in neutrophils isolated from the female is higher than the cut-off level, this is indicative that the female is suffering from or is at risk for developing GDM.

In some embodiments the level of myeloperoxidase and neutrophil elastase in the serum or plasma sample isolated from the female is compared to a cut-off level and to a control sample from a healthy pregnant female,

wherein if the level of myeloperoxidase in serum isolated from said female is higher than the cut-off level and the level of neutrophil elastase in serum isolated from said female is comparable to serum isolated from the control sample from a healthy pregnant female, this is indicative that said female is suffering from or is at risk for developing GDM;
wherein if the level of myeloperoxidase in plasma isolated from said female is higher than the cut-off level and the level of neutrophil elastase in serum isolated from said female is comparable to serum isolated from a control sample from the healthy pregnant female, this is indicative that said female is suffering from or is at risk for developing GDM.

In some embodiments a method for diagnosing gestational diabetes mellitus (GDM) in a pregnant female is provided, said method comprising detecting the level of polymorphonuclear neutrophils (PMN) and peripheral blood mononuclear cells (PBMC) in a blood sample isolated from said female, wherein the ratio of PMN to PBMC in the serum or plasma sample is indicative whether the female is suffering from or whether the female is at risk for developing GDM.

The level of polymorphonuclear neutrophils (PMN) and peripheral blood mononuclear cells (PBMC) in a serum or plasma sample isolated from said female is usually measured by cell counts. The ratio of PMN to PBMC (PMN/PBMC) is usually calculated based on the cell counts or as percentage of cells. Preferably the ratio of PMN to PBMC in the blood sample is indicative whether the female is suffering from or whether the female is at risk for developing GDM if the ratio of PMN to PBMC (PMN/PBMC) is between 3.0 and 8.0, preferably between 4.0 and 6.0 when calculated on the cell counts or if the ratio of PMN to PBMC (PMN/PBMC) is between 3.0 and 8.0, preferably between 5.0 to 7.0 when calculated on the percentage of cells.

In some embodiments a method for predicting or monitoring the efficacy of a therapeutic agent for use in treating GDM in a pregnant female is provided, said method comprising detecting the level of at least one biomarker selected from the group consisting of cell-free DNA, cell-free nucleosomes, myeloperoxidase and alpha-1 antitrypsin in a serum or plasma sample isolated from a female pre and post treatment with said therapeutic agent, wherein a change in the level of at least one of said biomarker indicates efficacy of said agent.

In some embodiments a method for the selection of a therapeutic agent for use in treating GDM in a pregnant female, comprising detecting NETosis in a serum or plasma sample isolated from said female is provided, wherein the level of NETosis in the serum or plasma sample is indicative whether circulatory polymorphic neutrophils (PMN) are used as target for the therapeutic agent. NETosis can be detected in a serum or plasma sample e.g. by detecting the level of biomarkers, by microscopic analysis of changes undergone by isolated PMN in in vitro culture, or by examination of PMN cultures for NETs products. Usually NETosis is detected by detecting the level of at least one biomarker. The at least biomarker is preferably selected from the group consisting of cell-free DNA, cell-free nucleosomes, myeloperoxidase and alpha-1 antitrypsin in the serum or plasma sample isolated from the female.

The therapeutic agent of the present invention may comprise a pharmaceutical composition, a signaling molecule, cells, manipulated cells, or combinations thereof. The pharmaceutical composition may include a known drug used for treating GDM such as e.g. insulin or oral hypogalecimic agents such as e.g. metformin.

In some embodiments a method for differentiating gestational diabetes mellitus (GDM) from auto-immune type I diabetes mellitus (T1DM) and/or from non-auto-immune type II diabetes mellitus (T2DM) in a pregnant female, said method comprising detecting at least one biomarker selected from the group consisting of cell-free DNA, cell-free nucleosomes, myeloperoxidase, neutrophil elastase, alpha-1 antitrypsin and DNaseI in a serum or plasma sample isolated from said female, wherein the level of at least one of said biomarker in the serum or plasma sample is indicative whether the female is suffering from or whether the female is at risk for developing GDM and/or whether the female is suffering from auto-immune type I diabetes mellitus (T1DM) or from non-auto-immune type II diabetes mellitus (T2DM). Preferably cell-free nucleosomes, myeloperoxidase, neutrophil elastase, alpha-1 antitrypsin and DNAseI are used as biomarkers. GDM can be differentiated from auto-immune type I diabetes (T1DM) and/or from non-auto-immune type II diabetes mellitus (T2DM) when:
i. In cases with GDM, the level of serum cell-free nucleosomes are highly increased compared to pregnant controls, while the level of serum myelopoxidase is moderately increased compared to pregnant controls and the level of serum neutrophil elastase is comparable to pregnant controls.
ii. In cases with T1DM, the level of serum cell-free nucleosomes is comparable to pregnant controls, while levels of both serum myeloperoxidase and neutrophil elastase are highly increased.
iii. In cases with T2DM, no increase of the level of serum cell-free nucleosomes, the level of serum myelopoxidase and the level of serum neutrophil elastase compared to pregnant controls are noted.

The surprising finding that excessively high levels of NETosis can occur in pregnant females can be used for predicting the risk for developing a disorder selected from the group consisting of preeclampsia, thrombotic events and cardiovascular damage in a pregnant female as these conditions have been associated with overt NETosis. Thus in some embodiments a method for predicting the risk for developing a disorder selected from the group consisting of preeclampsia, thrombotic events and cardiovascular damage, preferably a disorder selected from the group consisting of thrombotic events and cardiovascular damage, in a pregnant female is provided, said method comprising detecting NETosis in a serum or plasma sample isolated from said female, wherein the level of NETosis in the serum or plasma sample is indicative whether said female is at risk for developing preeclampsia, thrombotic events such as e.g. deep vein thrombosus or cardiovascular damage such as e.g. endothelial cell damage. In the method for predicting the risk for developing a disorder selected from the group consisting of preeclampsia, thrombotic events and cardiovascular damage, preferably a disorder selected from the group consisting of thrombotic events and cardiovascular damage, in a pregnant female NETosis can be detected in a serum or plasma sample e.g. by detecting the level of biomarkers, by microscopic analysis of changes undergone by isolated PMN in vitro culture, or by examination of PMN in in vitro culture supernatants for presence of NETosis products. Usually NETosis is detected by detecting the level of at least one biomarker. The at least biomarker is usually selected from the group consisting of cell-free DNA, cell-free nucleosomes, myeloperoxidase, alpha-1 antitrypsin and citrullinated histone H3 and is preferably selected from the group consisting of cell-free DNA, cell-free nucleosomes, myeloperoxidase, neutrophil elastase and alpha-1 antitrypsin in the serum or plasma sample isolated from the female.

In light of the findings that particular biomarkers can be used in diagnosing, predicting, and/or monitoring of GDM and/or for selecting a therapeutic agent for use in treating GDM the present invention also provides in some embodiments a composition comprising one or more reagents which binds specifically with and/or detects cell-free DNA, cell-free nucleosomes, myeloperoxidase and/or alpha-1 antitrypsin in a serum or plasma sample isolated from a female for use in diagnosing, predicting, and/or monitoring of GDM and/or for selecting a therapeutic agent for use in treating GDM.

The reagent which binds specifically with and/or detects cell-free DNA can be selected from the group consisting of fluorescent DNA bindings dyes (such as QuantiFluor® dsDNA System, Promega) and primers for methods which allow enumeration of DNA molecules by PCR based approaches such as real-time quantitative PCR or digital PCR, massive parallel sequencing (also termed next generation sequencing/NGS) or single molecule real time sequencing (also termed SMRT) approaches.

The reagent which binds specifically with and/or detects cell-free nucleosomes can be selected from the group consisting of antibodies and aptamers which detect histone molecules or the DNA fragments associated therewith thereby permitting detection of complete nucleosomes;

The reagent which binds specifically with and/or detects myeloperoxidase can be selected from the group consisting of antibodies, aptamers, and agents used to detect myeloperoxidase activity biochemically using calorimetry, an example being via the consumption of 5-thio-2-nitrobenzoic acid (TNB) using a taurine intermediate generated by enzyme activity of myeloperoxidase.

The reagent which binds specifically with and/or detects neutrophil elastase can be selected from the group consisting of antibodies, aptamers, and agents used to detect neutrophil elastase.

The reagent, which binds specifically with and/or detects alpha-1 antitrypsin can be selected from the group consisting of antibodies or aptamers, or agents used to detect alpha-1 antitrypsin activity biochemically by its ability to block the enzyme activity of proteases such as neutrophil elastase.

In light of the findings that particular biomarkers can be used in diagnosing, predicting, and/or monitoring of GDM and/or for selecting a therapeutic agent for use in treating GDM the present invention also provides in some embodiments a composition comprising one or more reagents which bind specifically with and/or detects citrullinated histone H3 in neutrophils isolated from a female for use in diagnosing, predicting, and/or monitoring of GDM and/or for selecting a therapeutic agent for use in treating GDM.

The reagent, which binds specifically with and/or detects citrullinated histone H3 can be selected from the group consisting of antibodies, aptamers, and agents used to detect citrullinated histone H3.

The nucleosome is a unit consisting of approximately 200 bases pairs of chromosomal double strand DNA complexed with histones H1, H2A, H2B, H3 and H4. Nucleosomes form the basic unit of the structure of chromosomes in the nucleus of eukaryotic cells. During compromise of the integrity of the cell plasma and nuclear membranes, chromosomes may gain access to the extracellular space and lose connection to the cell of origin. Due to cleavage by DNAses, the DNA strand is severed at the intersection between nucleosomal units, releasing nucleosomes of many different units, thus becoming cell-free nucleosomes. In the present disclosure, the terms “cell-free nucleosome(s)”, “cell-free DNA/histone complex” or “cell-free DNA/nucleosome complex” are used interchangeably. The detection of the cell-free nucleosomes in the serum or plasma sample isolated from said subject according to the methods of the present invention comprises contacting the serum or plasma sample with one or more reagents for detection of the cell-free nucleosomes and detecting specific binding of the one or more reagents with the cell-free nucleosomes. In one embodiment, the one or more reagents binds specifically with a histone or modified histone protein and with a DNA or modified DNA of the cell-free nucleosome and wherein specific binding of the one or more reagents to the histone or modified histone protein and to DNA or modified DNA of the cell-free nucleosome is indicative of the presence of GDM. Optionally, the one or more reagents binds specifically with a histone protein and modified DNA of the cell-free nucleosome. Optionally, the one or more reagents bind specifically with a modified histone protein and DNA of the cell-free nucleosome.

In some embodiments, the detection of the cell-free nucleosome in a serum or plasma sample isolated from said subject according to the methods of present invention comprises contacting the serum or plasma sample with a first reagent and a second reagent, wherein one of said first or said second reagent binds specifically with a DNA or a modified DNA of the cell-free nucleosome and the other of said first or second reagent binds specifically with a histone protein or a modified histone protein of the cell-free nucleosome. Optionally, one of said first or said second reagent binds specifically with a DNA of the cell-free nucleosome and the other of said or second reagent binds specifically with a modified histone protein of the cell-free nucleosome. Optionally, one of said first or said second reagent binds specifically with a modified DNA of the cell-free nucleosome and the other of said or second reagent binds specifically with a histone protein of the cell-free nucleosome. Optionally, detecting the cell-free nucleosomes according to any of the preceding embodiments further comprises immobilizing or fixing the cell-free nucleosomes or the modified histones of the cell-free nucleosomes on a solid such as microplate module suspended in a liquid. The said immobilization can be performed by any method, on any solid, or in any liquid known in art. The one or more reagents or the first and second reagent of the preceding embodiments can be detectably labeled.

The one or more reagents or the first and second reagent of the present invention and according to any disclosed embodiment is, preferably, an antibody, an aptamer, series of antibodies or aptamers, or combinations thereof.

The modified histone protein of the cell-free nucleosome according to the present invention and any disclosed embodiment has, preferably, a modification selected from histone deimination, histone methylation, histone phosphorylation or histone acetylation. More preferably, the histone deimination is histone 3 (H3), histone H2A (H2A) and/or histone H4 (H4) deimination.

The terms “citrullination” or “deimination” are used herein interchangeably. These terms denote to the post-translational modification of the amino acid arginine in a protein into the amino acid citrulline. “Citrullination” or “deimination” involves removal of an amine group from arginine, converting it to citrulline. This reaction is performed by enzymes called peptidylarginine deiminases (PADs). Note that citrullination of proteins is distinct from the formation of the free amino acid citrulline as part of the urea cycle or as a byproduct of enzymes of the nitric oxide synthase family. The modified DNA of the cell-free nucleosome according to the present invention and any disclosed embodiment, preferably, has a modification selected from DNA acetylation or DNA demethylation.

In another embodiment, detecting the cell-free nucleosomes according to any of the preceding embodiments further comprises immobilizing the cell-free nucleosomes or the modified histones of the cell-free nucleosomes on a solid suspended in a liquid. In another embodiment, said one or more reagents or said first and second reagent according to any of the preceding embodiments is detectably labeled. In another embodiment, said one or more reagents or said first and second reagent according to any of the preceding embodiments is an antibody, an aptamer, series of antibodies or aptamers, or combinations thereof.

In some embodiments a kit comprising the composition mentioned above is provided by the present invention. The term “kit” as used herein refers to a collection of reagents such as provided by the composition mentioned supra, preferably, provided in separately or within a single container. The container, also preferably, comprises instructions for carrying out the methods of the present invention.

A further embodiment of the present invention comprise the use of the methods or the kit as described supra in diagnosing, predicting, and/or monitoring of GDM and/or in selecting a therapeutic agent for use in treating GDM in a female.

In another embodiment the present invention provide the use of cell-free DNA, cell-free nucleosomes, myeloperoxidase, alpha-1 antitrypsin and/or citrullinated histone H3 or of cell-free DNA, cell-free nucleosomes, myeloperoxidase, and/or alpha-1 antitrypsin, in diagnosing, predicting, and/or monitoring of GDM and/or in selecting a therapeutic agent for use in treating GDM in a female.

### EXAMPLES

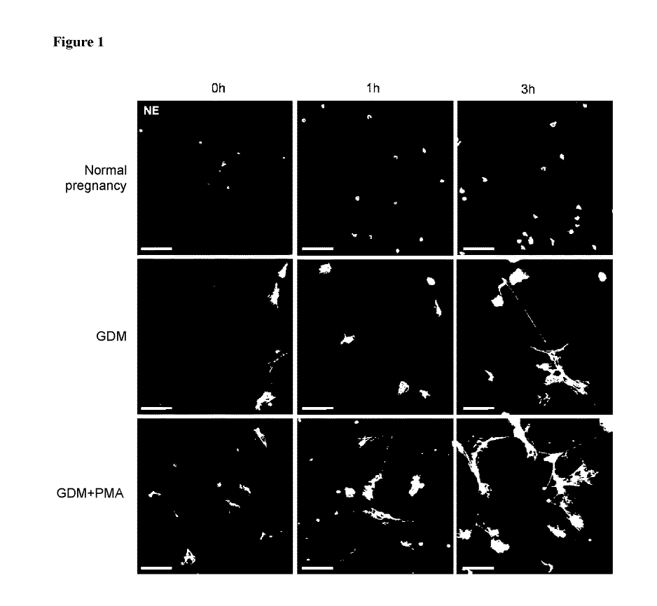

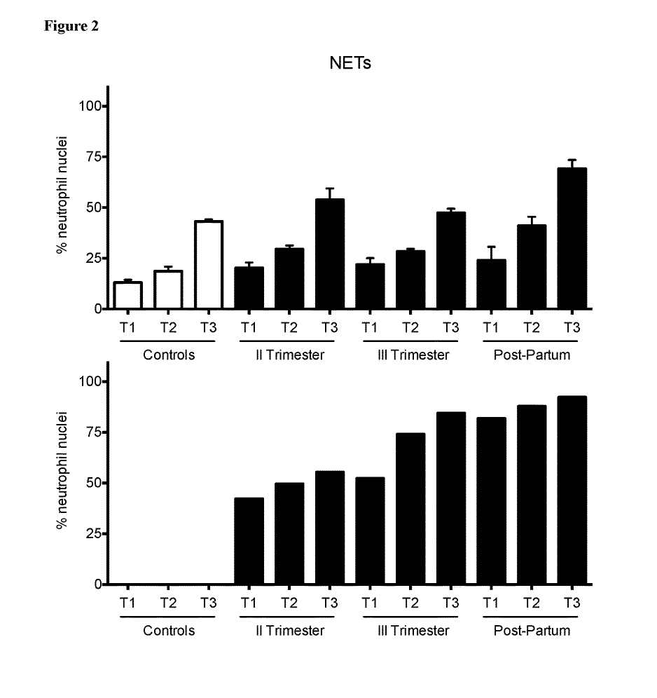

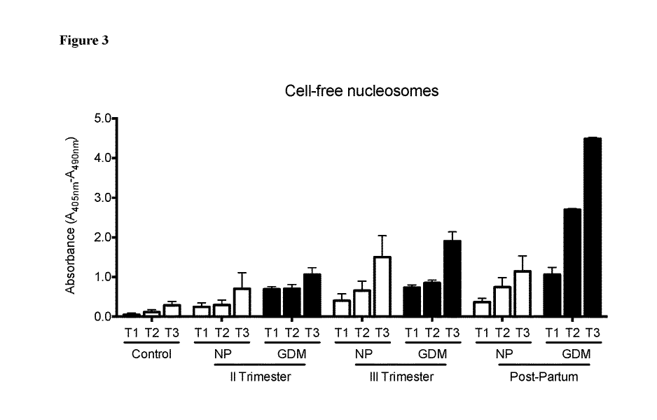

### Example 1: Isolation of Circulatory Neutrophils, Detection of NETs and Morphometric Analysis (FIGS. 1 and 2)

### Cell Isolation

Neutrophils were isolated by Dextran-Ficoll density centrifugation from EDTA or heparin venous blood samples as previously described [11]. In brief, peripheral blood was obtained by venipuncture and collected in EDTA-containing tubes (S-Monovette, Sarstedt). The collected blood was fractionated via density gradient centrifugation using Ficoll-Paque Plus (GE Healthcare) and neutrophils were isolated by dextran sedimentation of the RBC layer. Red blood cell lysis was performed with hypotonic salt solution. Circulating neutrophils were stained with trypan blue (MP Biomedical), to confirm cellular viability (96-98%), with a neutrophil population purity of >95%. Neutrophils seeded in 24-well plates were allowed to settle for 30 minutes at 37° C. under 5% CO₂ prior to further experimentation.

### Immunohistochemical Staining and Quantification of NETs

5×10⁴ isolated neutrophils were seeded on poly-L-lysine-coated glass coverslips (BD Biosciences) in tissue-culture wells and allowed to settle prior to stimulation as described above. Coverslips were rinsed with ice-cold HBSS and the cells fixed with 4% paraformaldehyde and blocked overnight (HBSS with 10% goat serum, 1% BSA, 0.1% Tween20, and 2 mM EDTA) at 4° C. NETs were detected with rabbit anti-NE (Abcam), rabbit anti-MPO (Dako), rabbit anti-PAD4 (Abcam), mouse anti-histone H1+core proteins (Millipore) and rabbit anti-citrullinated histone H3 (citH3, Abcam). Secondary antibodies were goat anti-rabbit IgG-AF555 and goat anti-rabbit IgG-AF488 (Invitrogen). DNA was stained with 4′,6-diamidino-2-phenylindole (DAPI, Sigma) and NETs were visualized using a Zeiss Axioplan 2 Imaging fluorescence microscope in conjunction with a Zeiss AxioCam MRm monochromatic CCD camera and analyzed with Axiovision 4.8.2 software (Carl Zeiss).

For enumeration and morphometric analysis, a minimum of 20 fields (at least 1000 neutrophils) per case was evaluated for myeloperoxidase/neutrophil elastase and DNA co-staining; nuclear phenotypes and NETs were counted and expressed as percentage of the total number of cells in the fields, using the ImageJ image analysis software (NIH) as described previously [12]. FIG. 1 shows excessive NETosis detectable in freshly isolated PMN from a case with GDM. FIG. 2 shows orphometric analysis as determined by fluorescent immunohistochemistry of isolated PMN in in-vitro culture indicating that the levels of spread (NET structures) are higher in cases with GDM from 2nd to 3rd trimester of pregnancy. This feature is especially evident post-partum.

### Example 2: Detection of Cell-Free Nucleosomes in Cell Culture Supernatants (FIG. 3)

Nucleosomes were measured using the Human Cell Death Detection ELISA^{PLUS} Kit® (Roche Diagnostics). Cell culture supernatants were incubated with 10U DNaseI for 5 min (Roche Diagnostics) prior to analysis, as described previously [12]. FIG. 3 shows increased NETosis by isolated PMN from GDM cases in various trimesters of pregnancy, as measured by detection of cell-free nucleosomes (Roche Cell Death ELISA Plus Kit®) in culture supernatants.

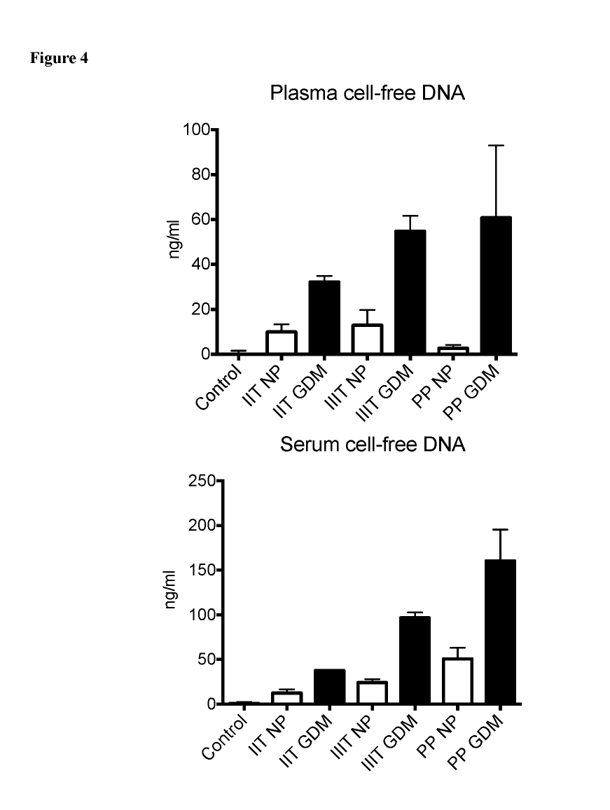

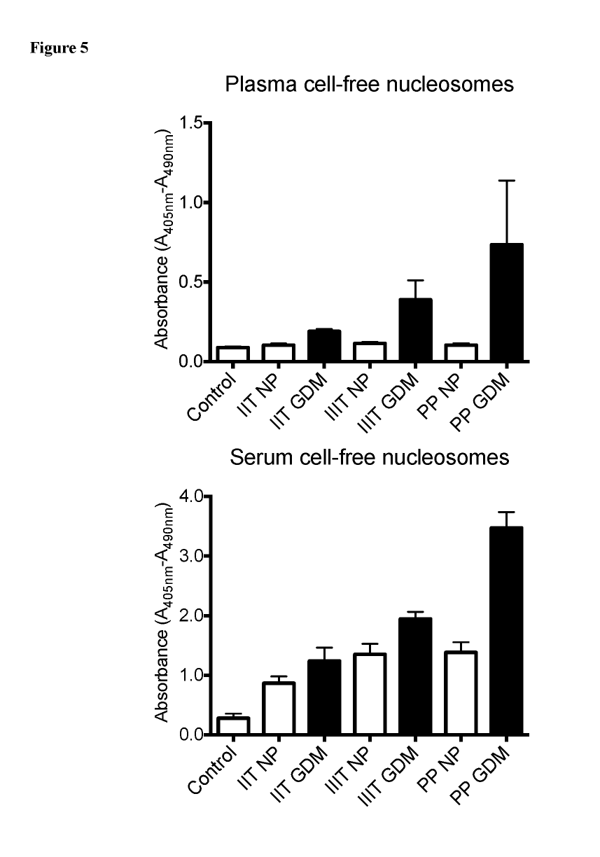

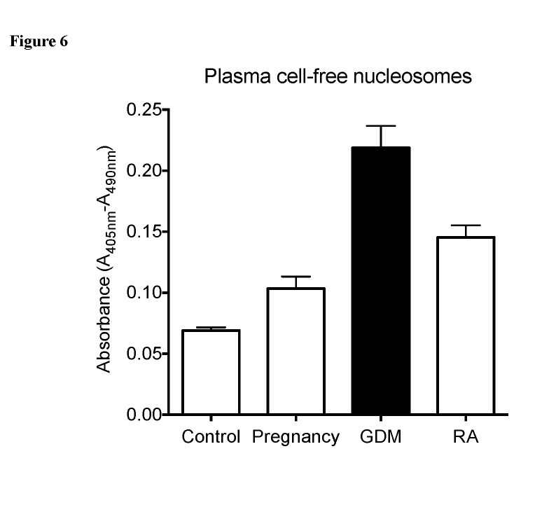

### Example 3: Detection of Cell-Free DNA in Plasma or Serum (FIG. 4)

### Preparation of Plasma and Serum

Plasma and serum was collected and processed as previously described [13,14]. In brief, peripheral blood was obtained by venipuncture and collected in EDTA- or heparin-containing tubes (S-Monovette, Sarstedt). Plasma and serum was separated from the cellular fractions through centrifugation after clotting or right after the blood collection, and samples were studied immediately or stored at −80° C. until analysis.

### Quantification of Cell-Free DNA by Binding to a Fluorescent Dye (Quantifluor® Promega)

Cell-free DNA in plasma or serum was quantified by fluorimetric detection via the use of a commercial assay (Quantifluor® Promega) according to manufacturer's instructions. A standard curve was used to determine the quantity of DNA detected as described in the manufacturer's instructions.

### Important Note—

Cell free DNA can also be quantified by:

### Cell Free DNA Isolation and Quantification by Real-Time PCR

Cell free DNA extracted from 850 μl plasma or serum using the QIAamp Circulating Nucleic Acid Kit (Qiagen) was quantified by TaqMan® real-time PCR (StepOne™ Plus Real-Time PCR System, Applied Biosystems) specific for the glyceraldehyde-3-phosphate dehydrogenase (GAPDH) gene [13,14].

In addition, it is possible to use digital PCR instead of real-time PCR, as this permits greater accuracy, or to use massive parallel sequencing or single molecule real time sequencing [15,16]. The advantage of the latter is that they are not locus limited but can be used to examine the entire genome [17]. FIG. 4 shows increased levels of cfDNA detected by fluorimetric assay (Promega Quantifluor® dsDNA System) directly in the freshly prepared plasma samples, as well as in corresponding serum samples.

### Example 4: Quantitation of Serum or Plasma Cell-Free Nucleosomes (FIGS. 5, 6 and 12)

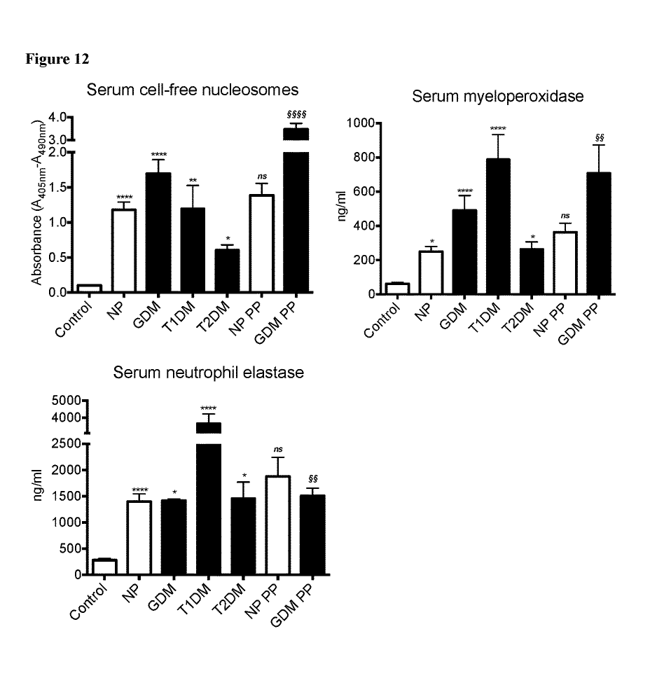

Plasma and serum was collected and processed as described previously [13,14]. Samples were studied immediately or stored at −80° C. until analysis. Nucleosomes were measured using the Human Cell Death Detection ELISA^{PLUS} Kit® (Roche Diagnostics), as described previously [12]. FIG. 5 shows increased levels of cell-free nucleosomes detected by commercial Roche Cell Death ELISA Plus Kit® directly in the freshly prepared plasma and corresponding serum samples. FIG. 6 shows increased levels of cell-free nucleosomes detected by commercial Roche Cell Death ELISA Plus Kit® in GDM plasma samples, compared to normal donors, healthy pregnant controls and RA cases. FIG. 12 shows increased levels of cell-free nucleosomes detected by commercial Roche Cell Death ELISA Plus Kit® in GDM and post-partum GDM serum samples, compared to normal donors, healthy pregnant and post-partum controls and cases with T1DM or T2DM.

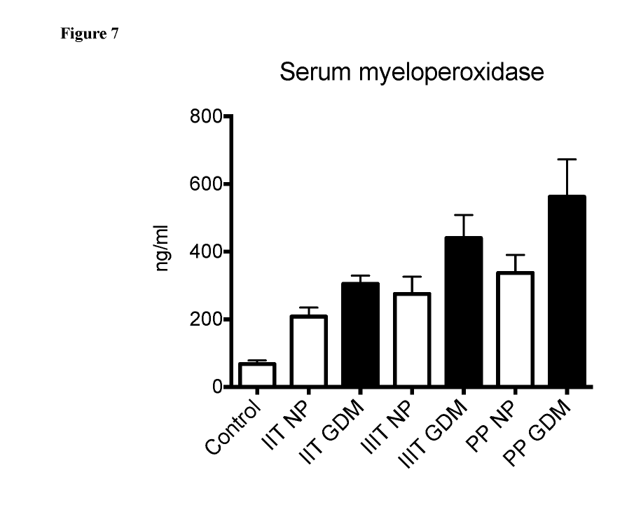

### Example 5: Quantitation of Serum or Plasma Myeloperoxidase or Neutrophil Elastase (FIGS. 7 and 12)

Plasma and serum was collected and processed as described previously [13,14]. The concentrations of neutrophil elastase (NE) and myeloperoxidase (MPO) were measured by sandwich ELISA (Elastase/a1-PI Complex ELISA Kit, Calbiochem) and the human MPO ELISA Kit (Hycult Biotech), respectively, as described previously [12]. FIG. 7 shows increased levels of myeloperoxidase (MPO) detected by commercial EIA in GDM serum samples. FIG. 12 shows increased levels of myeloperoxidase (MPO) detected by commercial sandwich ELISA in GDM and post-partum GDM serum samples, compared to normal donors, healthy pregnant and cases with T2DM. Levels of neutrophil elastase in GDM and post-partum GDM serum samples are comparable to healthy pregnant.

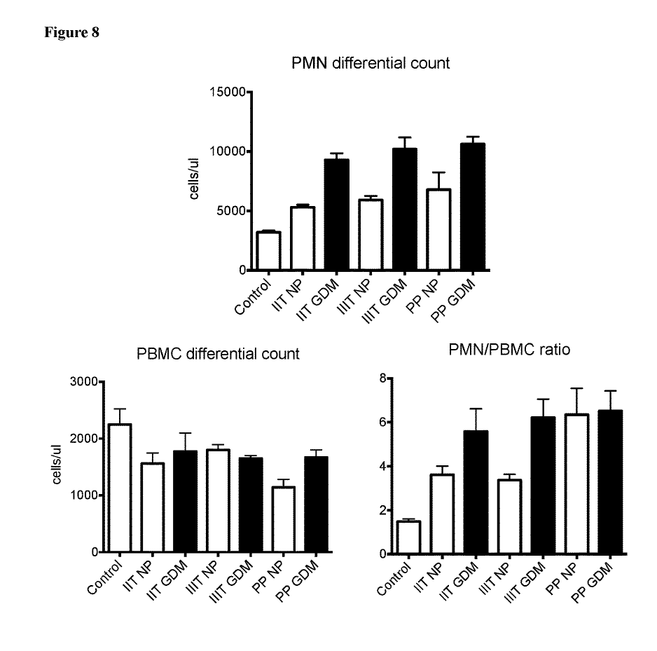

### Example 6: Complete Differential Blood Cell Count (FIG. 8)

Peripheral blood was obtained by venipuncture and collected in EDTA- or heparin-containing tubes (S-Monovette, Sarstedt). The complete differential blood cell count was performed utilizing an automated cellular analysis system (KX-21 N, Sysmex®), monitoring parameters such as total white blood cell differential count (WBC), neutrophil differential count (NEU), red blood cell differential count (RBC), platelet differential count (PLT) etc. FIG. 8 shows circulating levels of PMN that are elevated in cases with GDM, leading to an altered PMN/PBMC ratio.

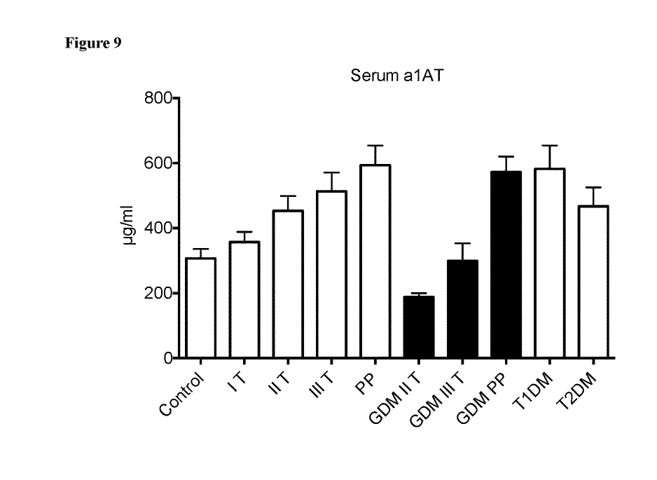

### Example 7: Quantitation of Serum or Plasma Alpha 1 Antitrypsin (FIG. 9)

Plasma and serum was collected and processed as described previously [13]. The concentration of alpha-1 antitrypsin (a1AT) was measured by sandwich ELISA (alpha 1 Antitrypsin (SerpinA1) Human ELISA kit, Abcam). FIG. 9 shows serum levels of a1AT that are reduced in cases with GDM when compared to normal healthy pregnancies. a1AT levels are not reduced in cases with T1DM or T2DM.

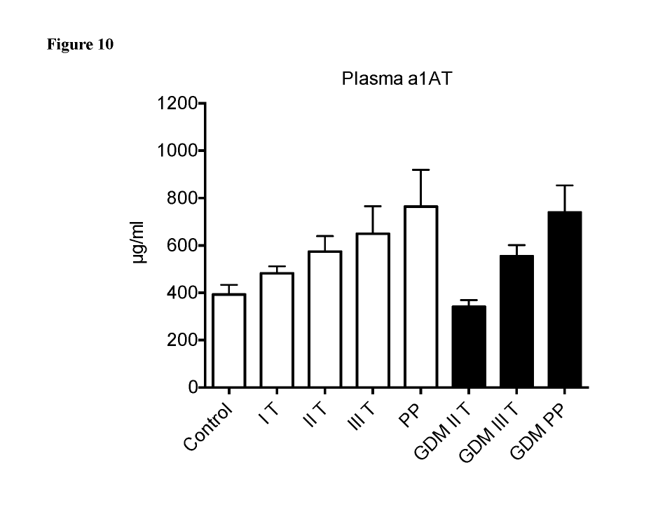

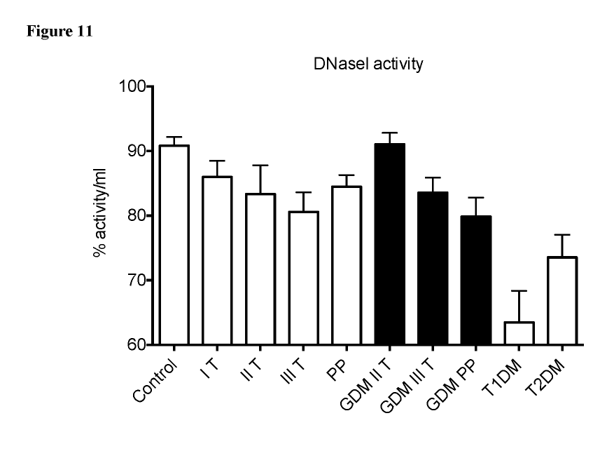

### Example 8: Quantitation of Serum or Plasma DNAse Activity (FIG. 10)

Plasma and serum was collected and processed as described previously [13]. Serum or plasma DNAse was assessed using a commercial Immunometric Enzyme Immunoassay for the Quantitative determination of DNase Activity (DNase Activity kit, Orgentec Diagnostika GmbH). FIG. 10 shows plasma levels of a1AT that are reduced in cases with GDM when compared to healthy normal pregnancies.

### Example 9: Detection of Nucleosome/Myeloperoxidase Complexes

MPO is present on extruded NETs. To detect such structures, NET associated MPO/DNA complexes were quantified utilizing a modified capture ELISA [18]. In brief, NET-associated MPO in serum or culture supernatant was captured using the coated 96 well plate of the human MPO ELISA Kit (Hycult Biotech), following which the NET-associated DNA backbone was detected using the detection antibody of the Human Cell Death Detection ELISA^{PLUS} Kit® (Roche Diagnostics), as described previously [12].

### Example 10: Detection of GDM at the Time of the Actual Diagnostic Procedure, i.e. the Oral Glucose Tolerance Test (OGTT)

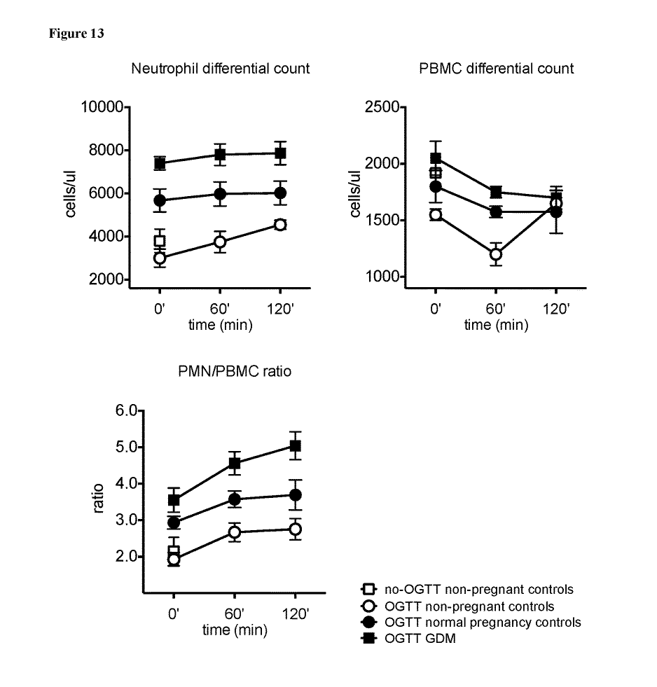

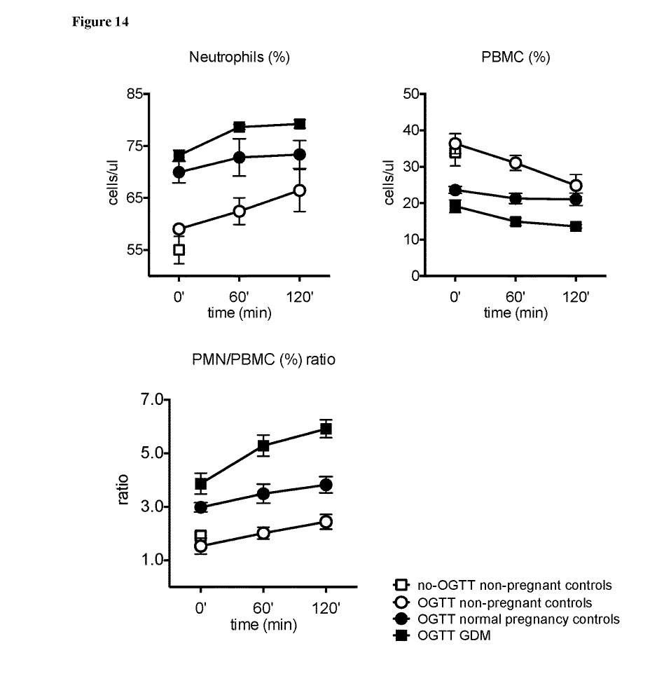

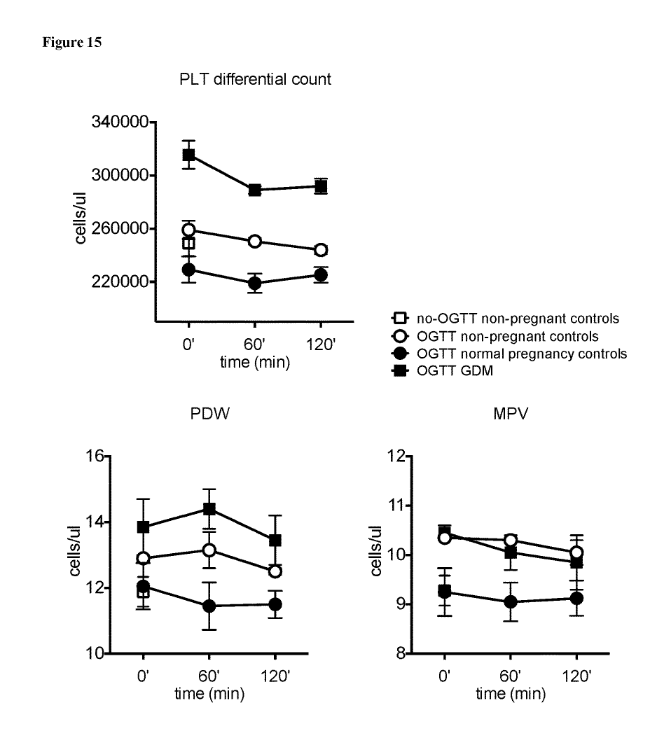

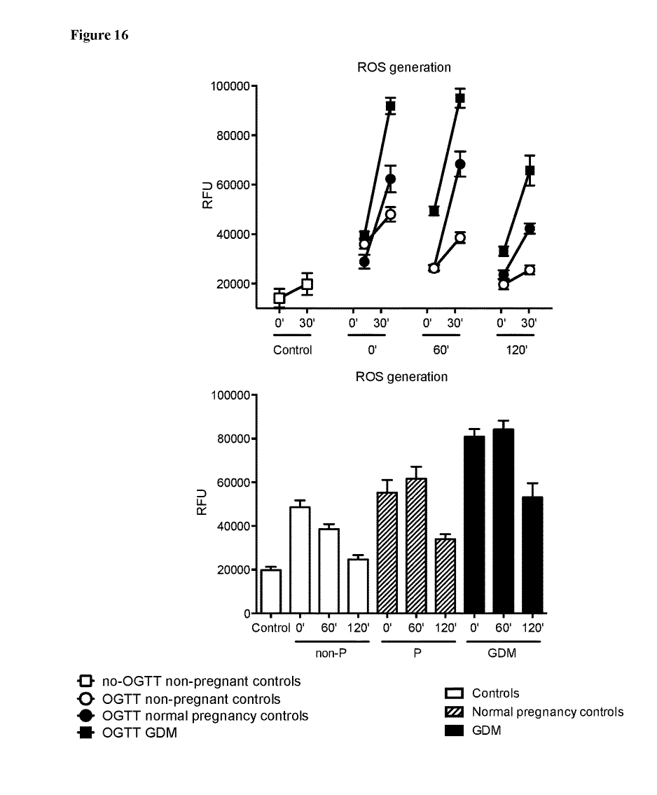

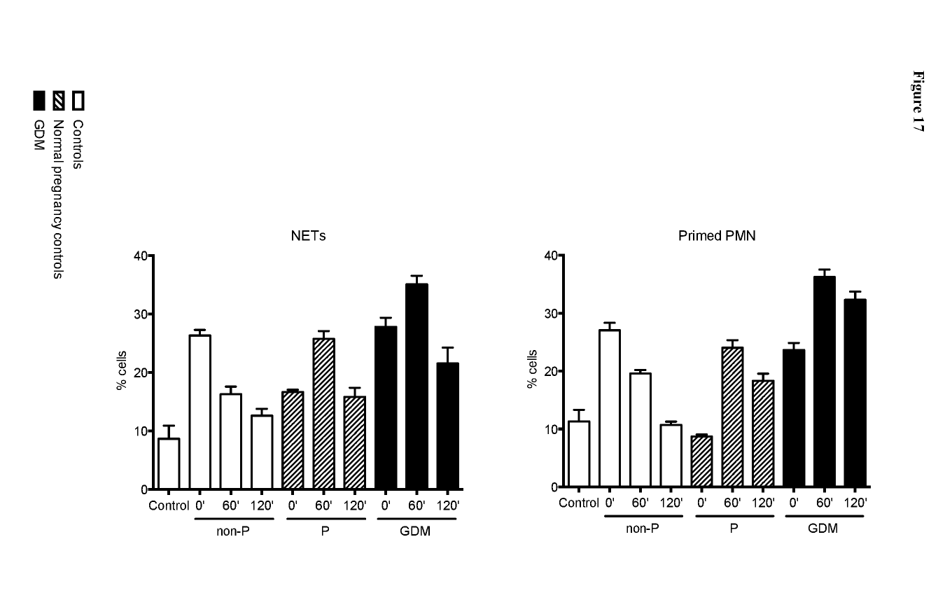

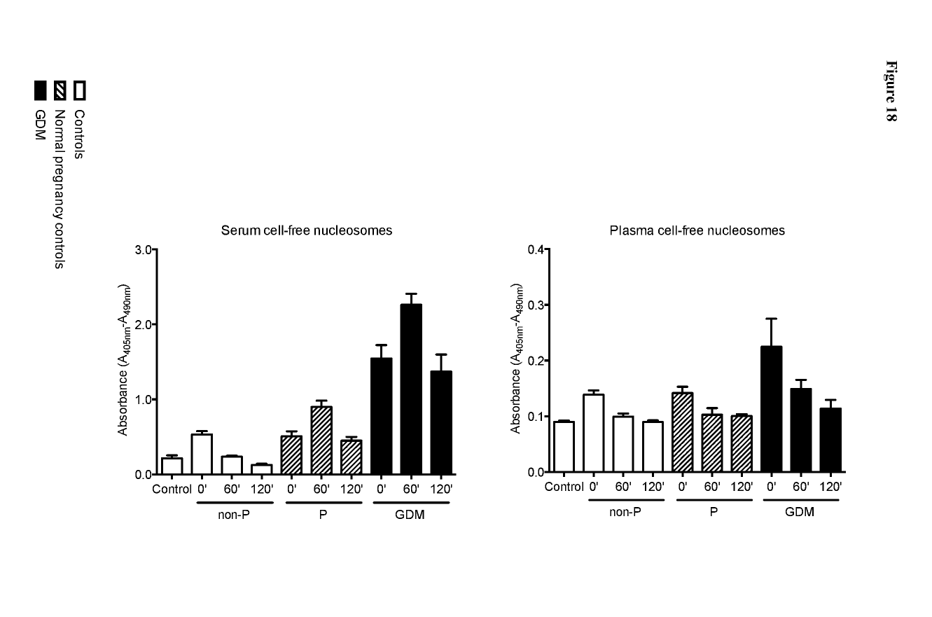

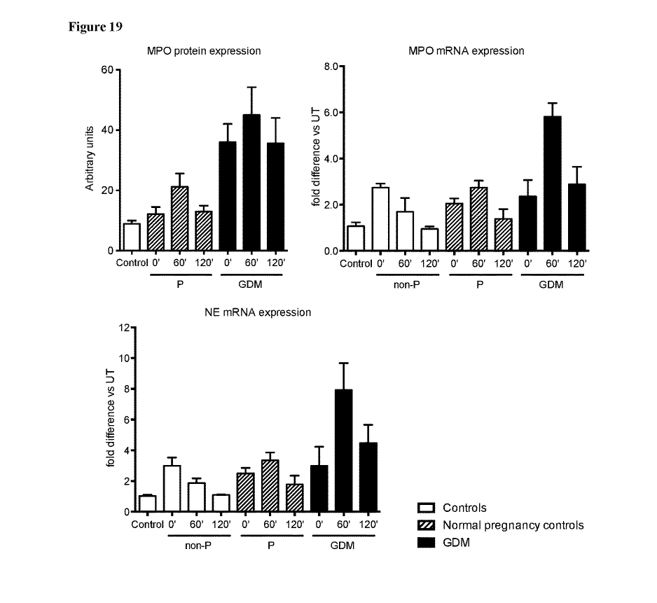

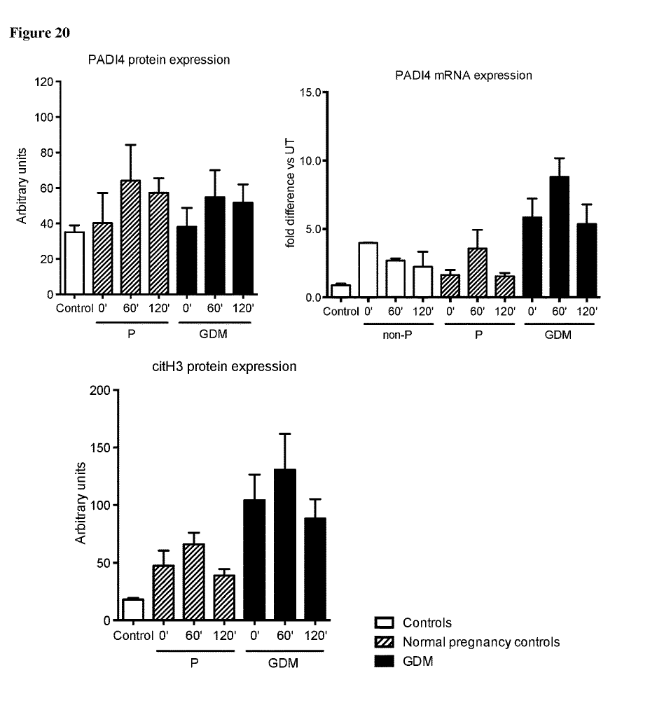

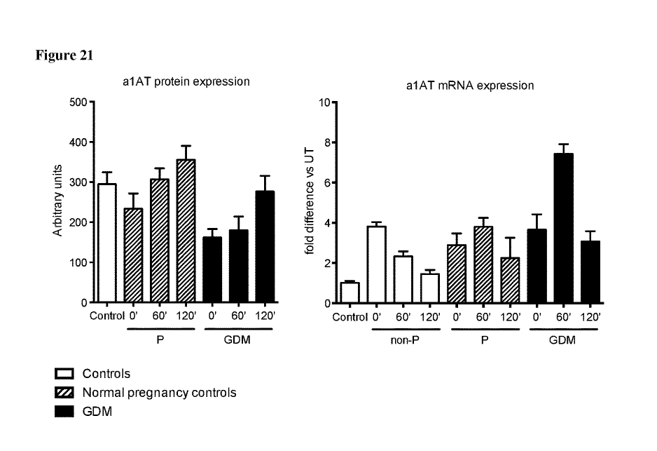

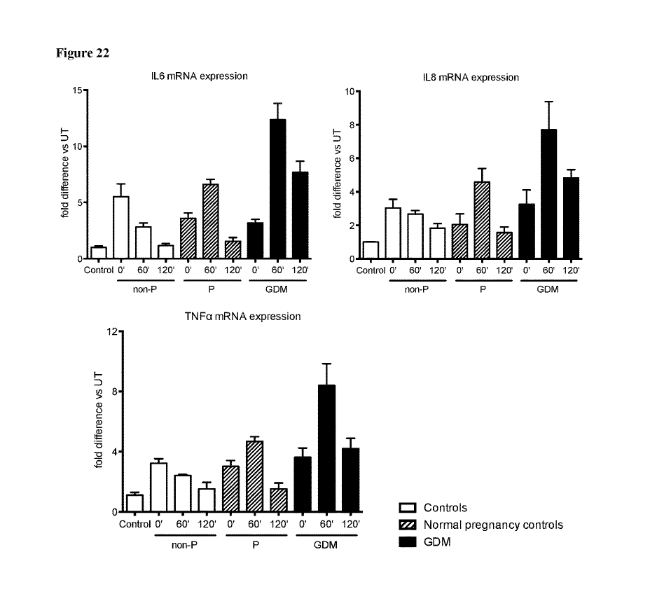

In order to evaluate the neutrophil activation state in gestational diabetes mellitus (GDM) patients and the direct effect of glucose on these neutrophils, 7 pregnant women and 3 non-pregnant healthy control individuals were recruited and blood was drawn at the time of the actual diagnostic procedure during a scheduled examination at the beginning of the second trimester (20-24 weeks of gestation), i.e. the oral glucose tolerance test (OGTT). During the OGTT glucose levels are monitored before, 60 min after and 120 min after consumption of 75 g glucose. Diagnosis of GDM is set when glucose levels are over the value of 8.0 60 min after glucose consumption and is still maintained over 8.0 after a period of two hours. Three out of the 7 pregnant women undergone the OGTT were diagnosed with GDM. In FIGS. 13, 14 and 15 all cell counts and all related cellular data are given. In GDM patients the neutrophil to PBMC ratio is the highest compared to matched non-pregnant controls and control pregnant individuals. Interestingly, platelet characteristics are lowest in cases with GDM. In FIGS. 16, 17, 18 and 19 data concerning ROS generation, morphometric analysis and cell free nucleosomes in serum an plasma are given. In all cases GDM patients show the highest values compared to non-pregnant controls and control individuals during their normal second trimester check-up. In FIGS. 20, 21 and 22 data concerning neutrophil protein and mRNA analysis is given. In all cases, GDM patients show the highest expression levels of the neutrophil markers NE, MPO, PADI4, citH3, a1AT and the proinflammatory cytokines IL6, IL8 and TNFa, as well as protein expression levels (with the exception of a1AT) compared to non-pregnant healthy controls and pregnant individuals with no GDM diagnosis. Interestingly, PADI4 protein shows similar patterns of expression between GDM and controls, while a1AT protein expression is lower in GDM. This latter feature agrees with previous data concerning a1AT expression levels in serum and plasma from GDM patients.

### CONCLUSIONS

NETosis and NETosis derived products such as the biomarkers described supra can act as a screening marker to detect cases with GDM. In this manner it can serve to optimise current screening protocols, and hopefully assist in reducing the very high false positive rate. In current tests up to 25% of all pregnant women can be rendered “screen positive”, leading to a clinical burden and possible “over diagnosis and treatment”. Our data suggest that optimal therapy for GDM must address the issue of overt PMN activity, and as such identify these cells as a key therapeutic target.

### REFERENCES

1. Buchanan T A, Xiang A, Kjos S L, Watanabe R (2007) What is gestational diabetes? Diabetes Care 30 Suppl 2: S105-111.
2. Sacks D B (2014) Diagnosis of gestational diabetes mellitus: it is time for international consensus. Clin Chem 60: 141-143.
3. Sacks D A (2009) Gestational diabetes—whom do we treat? N Engl J Med 361: 1396-1398.
4. Crowther C A, Hiller J E, Moss J R, McPhee A J, Jeffries W S, et al. (2005) Effect of treatment of gestational diabetes mellitus on pregnancy outcomes. N Engl J Med 352: 2477-2486.
5. Cundy T, Ackermann E, Ryan E A (2014) Gestational diabetes: new criteria may triple the prevalence but effect on outcomes is unclear. BMJ 348: g1567.
6. Landon M B, Spong C Y, Thom E, Carpenter M W, Ramin S M, et al. (2009) A multicenter, randomized trial of treatment for mild gestational diabetes. N Engl J Med 361: 1339-1348.
7. Kleinwechter H, Schafer-Graf U, Buhrer C, Hoesli I, Kainer F, et al. (2014) Gestational Diabetes Mellitus (GDM) Diagnosis, Therapy and Follow-Up Care. Exp Clin Endocrinol Diabetes 122: 395-405.
8. Moss J R, Crowther C A, Hiller J E, Willson K J, Robinson J S, et al. (2007) Costs and consequences of treatment for mild gestational diabetes mellitus—evaluation from the ACHOIS randomised trial. BMC Pregnancy Childbirth 7: 27.
9. Rowan J A, Hague W M, Gao W, Baffin M R, Moore M P, et al. (2008) Metformin versus insulin for the treatment of gestational diabetes. N Engl J Med 358: 2003-2015.
10. Rowan J A, Mi G I (2007) A trial in progress: gestational diabetes. Treatment with metformin compared with insulin (the Metformin in Gestational Diabetes [MiG] trial). Diabetes Care 30 Suppl 2: S214-219.
11. Gupta A K, Hasler P, Holzgreve W, Gebhardt S, Hahn S (2005) Induction of neutrophil extracellular DNA lattices by placental microparticles and IL-8 and their presence in preeclampsia. Hum Immunol 66: 1146-1154.
12. Chowdhury C S, Giaglis S, Walker U A, Buser A, Hahn S, et al. (2014) Enhanced neutrophil extracellular trap generation in rheumatoid arthritis: analysis of underlying signal transduction pathways and potential diagnostic utility. Arthritis Res Ther 16.
13. Zhong X Y, Laivuori H, Livingston J C, Ylikorkala O, Sibai B M, et al. (2001) Elevation of both maternal and fetal extracellular circulating deoxyribonucleic acid concentrations in the plasma of pregnant women with preeclampsia. Am J Obstet Gynecol 184: 414-419.
14. Zhong X Y, von Muhlenen I, Li Y, Kang A, Gupta A K, et al. (2007) Increased concentrations of antibody-bound circulatory cell-free DNA in rheumatoid arthritis. Clin Chem 53: 1609-1614.
15. Zimmermann B G, Grill S, Holzgreve W, Zhong X Y, Jackson L G, et al. (2008) Digital PCR: a powerful new tool for noninvasive prenatal diagnosis? Prenat Diagn 28: 1087-1093.
16. Hahn S, Lapaire O, Tercanli S, Kolla V, Hosli I (2011) Determination of fetal chromosome aberrations from fetal DNA in maternal blood: has the challenge finally been met? Expert Rev Mol Med 13: e16.
17. Chiu R W, Lo Y M (2013) Clinical applications of maternal plasma fetal DNA analysis: translating the fruits of 15 years of research. Clin Chem Lab Med 51: 197-204.
18. Kessenbrock K, Krumbholz M, Schonermarck U, Back W, Gross W L, et al. (2009) Netting neutrophils in autoimmune small-vessel vasculitis. Nat Med 15: 623-625.

1. A method for diagnosing gestational diabetes mellitus (GDM) in a pregnant female, said method comprising detecting NETosis in a serum or plasma sample isolated from said female, wherein the level of NETosis in the serum or plasma sample is indicative whether the female is suffering from or whether the female is at risk for developing GDM.

2. The method of claim 1, wherein NETosis is detected by detecting the level of at least one biomarker selected from the group consisting of cell-free DNA, cell-free nucleosomes, myeloperoxidase and alpha-1 antitrypsin in the serum or plasma sample isolated from said female.

3. A method for diagnosing gestational diabetes mellitus (GDM) in a pregnant female, said method comprising detecting citrullinated histone H3 in neutrophils isolated from said female, wherein the level of citrullinated histone H3 in the neutrophils is indicative whether the female is suffering from or whether the female is at risk for developing GDM.

4. The method according to claim 3, wherein the level of citrullinated histone H3 in neutrophils isolated from said female is compared to the level of citrullinated histone H3 in neutrophils of a control sample isolated from a healthy female, wherein a higher level of citrullinated histone H3 in neutrophils isolated from said female compared to the level of citrullinated histone H3 in neutrophils of the control sample is indicative that said female is suffering from or is at risk for developing GDM.

5. A method for diagnosing gestational diabetes mellitus (GDM) in a pregnant female, said method comprising detecting at least one biomarker selected from the group consisting of cell-free DNA, cell-free nucleosomes, myeloperoxidase and alpha-1 antitrypsin in a serum or plasma sample isolated from said female, wherein the level of at least one of said biomarker in the serum or plasma sample is indicative whether the female is suffering from or whether the female is at risk for developing GDM.

6. The method according to claim 2 or 5, wherein the level of the at least one biomarker in the serum or plasma sample isolated from said female is compared to a control serum or plasma sample isolated from a healthy female, wherein a higher level of cell-free DNA, cell-free nucleosomes or myeloperoxidase in the serum or plasma sample isolated from said female compared to the control serum or plasma sample is indicative that said female is suffering from or is at risk for developing GDM, and wherein a lower level of alpha-1 antitrypsin in the serum or plasma sample isolated from said female compared to the control serum or plasma sample is indicative that said female is suffering from or is at risk for developing GDM.

7. The method according to claim 2 or 5, wherein the level of the at least one biomarker in the serum or plasma sample isolated from said female is compared to a cut-off level, wherein if the level of cell-free DNA in serum isolated from said female is higher than the cut-off level, this is indicative that said female is suffering from or is at risk for developing GDM;
wherein if the level of cell-free DNA in plasma isolated from said female is higher than the cut-off level, this is indicative that said female is suffering from or is at risk for developing GDM;
wherein if the level of cell-free nucleosomes in serum isolated from said female is higher than the cut-off level, this is indicative that said female is suffering from or is at risk for developing GDM;
wherein if the level of cell-free nucleosomes in plasma isolated from said female is higher than the cut-off level, this is indicative that said female is suffering from or is at risk for developing GDM;
wherein if the level of myeloperoxidase in serum isolated from said female is higher than the cut-off level, this is indicative that said female is suffering from or is at risk for developing GDM;
wherein if the level of myeloperoxidase in plasma isolated from said female is higher than the cut-off level, this is indicative that said female is suffering from or is at risk for developing GDM;
wherein if the level of alpha-1 antitrypsin in serum isolated from said female is lower than the cut-off level, this is indicative that said female is suffering from or is at risk for developing GDM; and/or
wherein if the level of alpha-1 antitrypsin in plasma isolated from said female is lower than the cut-off level, this is indicative that said female is suffering from or is at risk for developing GDM.

8. A method for diagnosing gestational diabetes mellitus (GDM) in a pregnant female, said method comprising detecting the level of polymorphonuclear neutrophils (PMN) and peripheral blood mononuclear cells (PBMC) in a blood sample isolated from said female, wherein the ratio of PMN to PBMC in the blood sample is indicative whether the female is suffering from or whether the female is at risk for developing GDM.

9. A method for predicting or monitoring the efficacy of a therapeutic agent for use in treating GDM in a pregnant female, said method comprising detecting the level of at least one biomarker selected from the group consisting of cell-free DNA, cell-free nucleosomes, myeloperoxidase and alpha-1 antitrypsin in a serum or plasma sample isolated from a female pre and post treatment with said therapeutic agent, wherein a change in the level of at least one of said biomarker indicates efficacy of said agent.

10. A method for differentiating gestational diabetes mellitus (GDM) from auto-immune type I diabetes (T1DM) and/or from non-auto-immune type II diabetes (T2DM) in a pregnant female, said method comprising detecting at least one biomarker selected from the group consisting of cell-free DNA, cell-free nucleosomes, myeloperoxidase, neutrophil elastase, alpha-1 antitrypsin and DNaseI in a serum or plasma sample isolated from said female, wherein the level of at least one of said biomarker in the serum or plasma sample is indicative whether the female is suffering from or whether the female is at risk for developing GDM and/or whether the female is suffering from auto-immune type I diabetes (T1DM) or from non-auto-immune type II diabetes (T2DM).

11. A method for predicting the risk for developing a disorder selected from the group consisting of preeclampsia, thrombotic events and cardiovascular damage in a pregnant female said method comprising detecting NETosis in a serum or plasma sample isolated from said female, wherein the level of NETosis in the serum or plasma sample is indicative whether said female is at risk for developing preeclampsia, thrombotic events or cardiovascular damage.

12. The method of claim 11, wherein the disorder is selected from the group consisting of thrombotic events and cardiovascular damage.

13. A method for the selection of a therapeutic agent for use in treating GDM in a pregnant female, comprising detecting NETosis in a serum or plasma sample isolated from said female, wherein the level of NETosis in the serum or plasma sample is indicative whether circulatory polymorphic neutrophils (PMN) are used as target for the therapeutic agent.

14. The method according to any one of claims 1-13, wherein the female is pregnant in the 1^{st} or 2^{nd} trimester.

15. A composition comprising one or more reagents which bind specifically with and/or detects cell-free DNA, cell-free nucleosomes, myeloperoxidase or alpha-1 antitrypsin in a serum or plasma sample isolated from a female for use in diagnosing, predicting, and/or monitoring of GDM and/or for selecting a therapeutic agent for use in treating GDM.

16. The composition according to claim 15, wherein the reagent which binds specifically with and/or detects cell-free DNA is selected from the group consisting of fluorescent DNA bindings dyes and primers for real-time quantitative PCR, digital PCR, next generation sequencing (NGS) or single molecule real time sequencing (SMRT);
wherein the reagent which binds specifically with and/or detects cell-free nucleosomes is selected from the group consisting of antibodies or aptamers which detect histone molecules or the DNA fragments associated therewith;
wherein the reagent which binds specifically with and/or detects myeloperoxidase is selected from the group consisting of antibodies or aptamers, or agents used to detect myeloperoxidase activity biochemically; or
wherein the reagent which binds specifically with and/or detects alpha-1 antitrypsin is selected from the group consisting of antibodies or aptamers, or agents used to detect alpha-1 antitrypsin activity biochemically.

17. A composition comprising one or more reagents which bind specifically with and/or detects citrullinated histone H3 in neutrophils isolated from a female for use in diagnosing, predicting, and/or monitoring of GDM and/or for selecting a therapeutic agent for use in treating GDM.

18. The composition according to claim 17, wherein the reagent which binds specifically with and/or detects citrullinated histone H3 is selected from the group consisting of antibodies, aptamers, and agents used to detect citrullinated histone H3.

19. A kit comprising the composition according to any one of claims 15-18.

20. Use of the method according to any one of claim 1-8, or 13 or the kit according to claim 19 in diagnosing, predicting, and/or monitoring of GDM and/or in selecting a therapeutic agent for use in treating GDM in a female.

21. Use of cell-free DNA, cell-free nucleosomes, myeloperoxidase, alpha-1 antitrypsin and/or citrullinated histone H3, in diagnosing, predicting, and/or monitoring of GDM and/or in selecting a therapeutic agent for use in treating GDM in a female.

22. The use according to claim 21, wherein cell-free DNA, cell-free nucleosomes, myeloperoxidase, and/or alpha-1 antitrypsin are used.
